(19) United States
(12) Patent Application Publication
(10) Pub. No.: US 2023/0181647 A1
(43) Pub. Date: Jun. 15, 2023

(54) TREATMENT OF OVARIAN FAILURE USING REGENERATIVE CELLS

(71) Applicant: FIGENE, LLC, Houston, TX (US)

(72) Inventor: Thomas ICHIM, San Diego, CA (US)

(21) Appl. No.: 17/998,380

(22) PCT Filed: May 14, 2021
(86) PCT No.: PCT/US2021/070555

§ 371 Date: Nov. 10, 2022

Related U.S. Application Data
(60) Provisional application No. 63/025,092, filed on May 14, 2020.

(51) Int. Cl. A61K 35/33; C12N 5/075; C12N 5/077

(52) U.S. Cl. CPC A61K 35/33; C12N 5/0609; C12N 5/0656; C12N 2500/02; C12N 2501/10; C12N 2506/1307

(57) ABSTRACT

Disclosed herein are methods and compositions for treating or preventing ovarian failure using fibroblasts or cells derived from fibroblasts. In some embodiments, ovarian failure is pathological, the result of an intervention, or the result of aging. In some embodiments, regenerative fibroblast cells are administered locally into the ovary or pen-ovary areas or systemically. In some embodiments, regenerative cells act to suppress fibrosis of the ovaries, inhibit inflammation, stimulate maturation of immature ovarian progenitor cells, or directly differentiate into oocytes. In some embodiments, regenerative fibroblasts produce factors that inhibit apoptosis of oocytes and/or oocyte progenitors.

This application claims priority to U.S. Provisional Patent Application Ser. No. 63/025,092, filed on May 14, 2020, which is incorporated by reference herein in its entirety.

### TECHNICAL FIELD

Embodiments of the disclosure generally include at least the fields of cell biology, molecular biology, gynecology, and medicine. More particularly, the disclosure pertains to the area of the female reproductive system and the use of fibroblasts for treatment of ovarian failure.

### BACKGROUND

Human health deteriorates as humans age because of impaired function of given organs, which in turn yields disorders specific to that organ, and breakdown of inter-organ communication networks controlled primarily by hormonal signals. The ovaries are a classic example of both situations. The female gonads, or ovaries, serve as the source of both germ cells (oocytes) needed for reproduction and a large number of bioactive factors that support or modulate the function of many other tissues and cells.

Oogenesis begins with the formation of primordial germ cells (PGC's), a source of adult germ cells. Primordial germ cells arise in the extra-embryonic tissues of the yolk sac and allantois, migrate into the hindgut epithelium and along the dorsal mesentary of the genital ridges, and finally arrive in the primitive gonad. The PGC's undergo approximately 7 to 8 mitotic divisions during migration and until 2 to 3 days after arrival in the ovary and are converted to oogonia, which are connected by intercellular bridges (cell syncytium) and begin actively dividing.

Oogonia become oocytes once they cease mitosis and enter meiosis. Meiosis continues until oocytes reach the dictyate stage of the first meiotic prophase, which is at or shortly after parturition in most species. During this stage, oocytes undergo a period of extensive growth and discontinue meiosis until the gonadotropin surge at ovulation. At that time, meiosis resumes and continues until oocytes are arrested at metaphase II (unfertilized oocytes). The beginning of meiotic reduction is also evidenced by first polar body extrusion. Oocytes remain at this stage until fertilization or parthenogenetic activation, at which time meiosis is completed and the second polar body is extruded.

The most dramatic aspect of oocyte growth is the 300-fold increase in size to become one of the largest cells in the body. During oocyte growth, distinct structural changes occur. These include an increase in the diameter of the nucleus (or germinal vesicle; GV) as well as a marked decrease in the nuclear to cytoplasm ratio; enlarged nucleoli, which also change from diffuse and granular to a dense and fibrillar network; an increase in the number of mitochondria as well as a change from elongated mitochondria with transverse cristae to round mitochondria with columnar cristae; a change in Golgi membranes from flat stacks of arched lamellae with no vacuoles to swollen stacks of lamellae with many vacuoles; the appearance of cortical granules; the appearance and growth of the zona pellucida; an increase in the number of ribosomes; and the appearance of cytoplasmic lattices.

Biochemical changes also occur during oocyte growth. An extremely large amount of total ribonucleic acid (RNA) at levels 200-fold higher than in somatic cells is present in murine oocytes, and protein synthesis and storage occurs at levels 50-fold higher than in somatic cells. RNA and protein accumulate primarily because cytokinesis does not occur, although the concentration of total RNA and protein are not different from somatic cells. Some specific proteins that are synthesized during murine oocyte growth are mitochondrial and ribosomal proteins, zona pellucida glycoproteins, histones, tubulin, actin, calmodulin, lactate dehydrogenase, creatine kinase, and glucose-6-phosphate dehydrogenase. Changes in specific gene expression have also been reported during oocyte growth for murine oocytes. These include the presence of Oct-3 messenger RNA (mRNA) in growing oocytes, an increase in the number of c-kit transcripts, an increase in transcription of m-ZP3, unusually high levels of lactate dehydrogenase activity in oocytes prior to meiotic maturation, and numerous others.

Meiotic maturation is defined as the progression from the dictyate stage of the first meiotic prophase to metaphase II. Oocytes acquire meiotic competence by obtaining the ability to progress from GV breakdown to metaphase I and then obtaining the ability to progress from metaphase I to metaphase II. Porcine oocytes from follicles with an average diameter of 3 mm have attained meiotic competence.

Meiotic maturation is composed of a number of structural changes. Probably the most obvious structural change is GV (or nuclear) breakdown. This is very evident in murine oocytes; however, this can only be seen via a nuclear stain in porcine oocytes. The next sequence of landmarks include chromosome condensation (transition from diffuse dictyate-stage to V-shaped, telocentric bivalent chromosomes), spindle formation, and first polar body extrusion. Throughout these events, a number of alterations in microtubule and microfilament structure occur. Other biochemical changes occur during meiotic maturation including a dramatic decrease in RNA levels, a decrease in intracellular methionine levels, and a decrease in protein synthesis. Certain regulatory molecules are also involved in meiotic maturation. Factors suggested to inhibit GV breakdown are cyclic adenosine monophosphate and regulators of its intracellular levels, calcium, calmodulin, steroids, gonadotropins, purines, protein inhibitors, and intercellular communication between cumulus cells and the oocyte.

Two hypotheses exist for the resumption of meiosis by luteinizing hormone (LH) at ovulation: loss of inhibitory input and positive stimuli. The loss of inhibitory input hypothesis suggests that inhibitory substances (e.g., cyclic adenosine monophosphate) produced by granulosa or cumulus cells maintain meiotic arrest, and the LH surge at ovulation may terminate communication between the follicle granulosa cells and cumulus cells or between cumulus cells and the oocyte resulting in the absence of this inhibitory stimulus to the oocyte. The positive stimuli theory suggests that LH may induce production of a substance (calcium, adenosine triphosphate, pyruvate) from granulosa or cumulus cells that directly causes the oocyte to resume meiosis.

Dramatic decreases in tubulin, actin, histone, ribosomal protein, lactate dehydrogenase, and zona pellucida glycoprotein synthesis rates also occur upon meiotic maturation, as well as phosphorylation changes in cell cycle control proteins. Additionally, changes in specific gene expression during meiotic maturation have been reported for murine oocytes. These include a decrease in c-mos transcription between metaphase I and II, the presence of Oct-3 mRNA in ovulated oocytes, a dramatic drop in m-ZP3 RNA levels at ovulation, the appearance of tissue-type plasminogen activator transcripts following GV breakdown, and a sharp decrease in lactate dehydrogenase levels during meiotic maturation.

An important cytoplasmic factor involved in meiotic maturation is a protein called MPF. Maturation (M-phase, mitosis, meiosis) promoting factor (MPF) is ubiquitous to all dividing yeast, invertebrate, amphibian, and mammalian cells, and it controls the transition from the G2 to mitosis phases of the cell cycle. Two subunits form the MPF complex, including a 34 kilodalton (kD) catalytic subunit (p34cdc2; a protein kinase) and a 45 kD regulatory subunit (cyclin B). Levels of p34cdc2 are constant, while cyclin levels fluctuate throughout the cell cycle. Immature oocytes contain an inactive precursor to MPF, and dephosphorylation of p34cdc2 at tyrosine and threonine residues results in MPF activation, which is required for GV breakdown. At the end of metaphase I prior to first polar body extrusion, the cyclins are degraded, rendering the MPF complex inactive. New cyclins are synthesized, and MPF becomes highly active during metaphase II. Levels of MPF remain high during metaphase II due to a protein called cytostatic factor (CSF). This protein contains products of the c-mos (pp 39 mos, a 39 kD phosphoprotein) and cdk-2 (cyclin-dependent kinase 2) genes and appears to act by preventing cyclin degradation. Upon oocyte activation, CSF is destroyed by a protease that is activated by the release of Ca²⁺ ions and MPF levels drop, allowing meiosis completion and pronuclear formation. Examination of histone H1 kinase is used as an indication of MPF activity because p34cdc2 has been shown to phosphorylate histone H1 in vitro. These phosphorylation events have been used as a biochemical assay for the estimation of p34cdc2 activity.

The successful in vitro development of the oocyte has become much more important in recent years with the advances in molecular biology and an increased push for the production of transgenic animals. In vitro oocytes may also be helpful in treating ovarian failure. For reasons not fully known, the ovaries are the first major organs to fail in aging females, and this occurs long before age-related dysfunction of other tissues is observed. For example, in women, fertility becomes severely compromised around the age of forty, preceding the menopause by about a decade. Female mice exhibit a similar impairment of fertile potential approximately halfway through their chronological lifespan. Irrespective of the species evaluated, ovarian failure and the ensuing loss of the follicular ovarian reserve is a characteristic of the aging process. Perhaps even more important than the loss of fertility, age-related ovarian failure sets the stage in aging females for markedly increased risks of developing a large number of debilitating health issues, including osteoporosis, cardiovascular disease, and cognitive dysfunction.

In fact, recent studies in mice have solidified a direct causal association between ovarian failure and deteriorating health in aging females. For example, inactivation of the pro-apoptotic Bax gene sustains the follicle pool and thus functional ovarian lifespan into very advanced age [8, 9], which extends fertile potential in aging females and minimizes the onset of many age-related health problems, including bone and muscle loss, excess fat deposition, alopecia, cataracts, deafness, increased anxiety, and selective attention deficit. Other studies have demonstrated that overall lifespan can be increased by transplanting young adult ovaries into aging female mice.

For example, in one experiment, scientists investigated the capacity of young ovaries, transplanted into old ovariectomized CBA mice, to improve remaining life expectancy of the hosts. Donor females were sexually mature 2-month-olds; recipients were prepubertally ovariectomized at 3 weeks and received ovarian transplants at 5, 8 or 11 months of age. Relative to ovariectomized control females, life expectancy at 11 months was increased by 60% in 11-month recipient females and by 40% relative to intact control females. Only 20% of the 11-month transplant females died in the 300-day period following ovarian transplantation, whereas nearly 65% of the ovariectomized control females died during this same period. The 11-month-old recipient females resumed oestrus and continued to cycle up to several months beyond the age of control female reproductive senescence. Across the three recipient age groups, transplantation of young ovaries increased life expectancy in proportion to the relative youth of the ovary [10].

Despite the compelling nature of these findings, the infeasibility of similar approaches in humans has made possible clinical translation of this work uncertain. New data, however, suggests that ovarian function and fertility can be dramatically altered by technologies that might prove amenable for potential clinical development. Accordingly, there is need for a means of regenerating germ or gamete-like cells or components thereof in a natural and physiological manner to treat or prevent ovarian failure. The present disclosure provides solutions to long-felt needs in the art of treating or preventing ovarian failure.

### BRIEF SUMMARY

The present disclosure is directed to systems, methods, and compositions for the treatment and prevention of ovarian failure through administration of fibroblasts, regenerative fibroblasts, or fibroblast- and regenerative fibroblast-derived products. The disclosure is related to the area of fertility and reproductive biology. More particularly, the disclosure deals with methods of suppressing inflammation, stimulating maturation of immature oocytes, and inducing folliculogenesis.

In one embodiment, provided is a method of treating or preventing ovarian failure in an individual comprising administering a therapeutically effective amount of a composition comprising fibroblasts or conditioned media therefrom to an individual in need thereof. The ovarian failure can be age-related, idiopathic premature ovarian failure, and/or associated with treatment including but not limited to chemotherapy, radiation therapy, or a combination thereof.

In some embodiments, the fibroblasts comprise regenerative fibroblasts which are cultured under conditions sufficient to differentiate the fibroblasts into regenerative fibroblast cells. The regenerative fibroblast cells can comprise one or more of the following biological activities: (a) inducing of angiogenesis; (b) producing trophic factors; (c) suppressing inflammation; (d) stimulating maturation of immature oocytes; and (e) inducing folliculogenesis. The regenerative fibroblast cells can be cultured under conditions sufficient to enhance the ability of the regenerative fibroblast cells to induce angiogenesis, produce trophic factors, suppress inflammation, stimulate maturation of immature oocytes, induce folliculogenesis, or a combination thereof. Culture conditions can comprise hypoxia, which can be sufficient to induce nuclear translocation of HIF-1 alpha. Culture conditions can further comprise treatment of the regenerative fibroblast cells with one or more growth factors, one or more differentiation factors, one or more dedifferentiation factors, or a combination thereof.

In some embodiments, the regenerative fibroblast cells express one or more markers selected from the group consisting of Oct-4, Nanog, Sox-2, KLF4, c-Myc, Rex-1, GDF-3, LIF receptor, CD105, CD117, CD344, Stella, and a combination thereof. In some embodiments, the regenerative fibroblast cells do not express one or more cell surface proteins selected from the group consisting of MHC class I, MHC class II, CD45, CD13, CD49c, CD66b, CD73, CD105, CD90, and a combination thereof. The regenerative fibroblast cells can have enhanced GDF-11 expression compared to a control or standard.

The fibroblast cells of the present disclosure are, or can be derived from, fibroblasts which can be isolated from umbilical cord, skin, cord blood, adipose tissue, hair follicle, omentum, bone marrow, peripheral blood, Wharton's Jelly, or a combination thereof. The fibroblasts can also be obtained from dermal fibroblasts, placental fibroblasts, adipose fibroblasts, bone marrow fibroblasts, foreskin fibroblasts, umbilical cord fibroblasts, hair follicle derived fibroblasts, nail derived fibroblasts, endometrial derived fibroblasts, keloid derived fibroblasts, or a combination thereof. The fibroblast cells can be autologous, allogeneic, or xenogeneic to the recipient and can be purified from bone marrow and/or purified from peripheral blood. In some embodiments, the regenerative fibroblast cells are isolated from peripheral blood of an individual who has been exposed to one or more conditions and/or one or more therapies sufficient to stimulate regenerative fibroblast cells from the individual to enter the peripheral blood of the individual. The conditions sufficient to stimulate regenerative fibroblast cells from the individual to enter the peripheral blood can comprise administration of G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA reductase inhibitors, small molecule antagonists of SDF-1, or a combination thereof. The therapies sufficient to stimulate regenerative fibroblast cells from the individual to enter the peripheral blood can comprise therapies including exercise, hyperbaric oxygen, autohemotherapy by ex vivo ozonation of peripheral blood, induction of SDF-1 secretion in an anatomical area outside of the bone marrow, or a combination thereof.

In some embodiments, the regenerative fibroblast cells are comprised of an enriched population of regenerative fibroblast cells. Enrichment is achieved in some embodiments by: (a) transfecting the cells with a vector comprising a fibroblast-specific promoter operably linked to a reporter or selection gene, wherein the reporter or selection gene is expressed, and (b) enriching the population of cells for cells expressing the reporter or selection gene. Enrichment is achieved in some embodiments by: (a) treating the cells with a detectable compound, wherein the detectable compound is selectively detectable in proliferating and non-proliferating cells, and (b) enriching the population of cells for proliferating cells. The detectable compound can be selected from a group comprising carboxyfluorescein diacetate, succinimidyl ester, and Aldefluor.

In some embodiments, the regenerative fibroblast cells are fibroblasts isolated as side population cells. The fibroblasts isolated as side population cells can be identified based on expression of the multidrug resistance transport protein (ABCG2). The fibroblasts isolated as side population cells can also be identified based on the ability to efflux intracellular dyes. The side population cells can be derived from tissues selected from the group consisting of pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, mesentery tissue, and a combination thereof.

In some embodiments, the fibroblast cells express CD39, Oct3/4, and/or CD73. The CD73-positive fibroblast cells can be cultured under hypoxic conditions, which can comprise, for example, from 0.1% oxygen to 10% oxygen for a period of 30 minutes to 3 days or 3% oxygen for 24 hours. Hypoxic conditions can also be chemically induced by, for example, culture in 1 μM-300 μM or 250 82 M cobalt (II) chloride for 1-48 hours or 24 hours. Hypoxia can induce upregulation of HIF-1α, which can be detected by expression of VEGF secretion. Hypoxia can also induce upregulation of CXCR4 on the fibroblast cells, which can promote homing of the fibroblast cells to an SDF-1 gradient. In some embodiments, the fibroblast cells are treated with a histone deactylase inhibitor, which can be sodium butyrate, valproic acid, or trichostatin A.

In some embodiments, the fibroblast cells are induced in culture to express Vasa, Dazl, Stella, Fragilis, or a combination thereof, and the fibroblast cells do not express GDF-9, zona pellucida proteins, HDAC6, SCP3, or a combination thereof. The fibroblast cells can also be mitotically competent and possess an XX karyotype.

In some embodiments, an individual with ovarian failure is treated with fibroblasts to increase the number and activity of T regulatory cells, and the fibroblast cells are capable of inducing generation of T regulatory cells and produce growth factors comprising FGF, VEGF, IGF-1, HGF, or a combination thereof. The T regulatory cells can express the transcription factors FoxP3 and/or Helios, can suppress fibrosis, and/or can produce interleukin-10 and/or interleukin-35. In some embodiments, activity of the T regulatory cells is augmented by manipulation of the individual's microbiome, which can occur via administration of Lactobacillus reuteri. Inosine may also be administered in combination with the fibroblast cells.

In some embodiments, the fibroblast cells are cultured with one or more agents capable of increasing expression of fibroblast PD-1 ligand. The agents can comprise TGF-β and/or soluble HLA-G, and the increased expression of fibroblast PD-1 ligand can be associated with induction of T regulatory cells upon binding of PD-1-expressing fibroblasts to naïve T cells. In some embodiments, the fibroblast cells induce generation of adaptive immune cells, and wherein the adaptive immune cells are capable of reprogramming ovarian cells to suppress inflammation. The adaptive immune cell can be a B regulatory cell and/or a T regulatory cell, and the T regulatory cells can express CD4 and CD25.

The fibroblast cells of the disclosure can be administered locally or systemically, and local administration can be inside the ovary, in the peri-ovary area, or a combination thereof. Administration of the fibroblast cells can stimulate production of oocytes and can be augmented by culture with factors including TPO, SCF, IL-1, IL-3, IL-6, IL-7, IL-11, flt-3L, G-CSF, GM-CSF, Epo, FGF-1, FGF-2, FGF-4, FGF-20, IGF, EGF, NGF, LIF, PDGF, BMPs, activin-A, VEGF, forskolin, glucocorticoids, or a combination thereof.

Also administered in some embodiments to the individual is a therapeutically effective amount of a composition comprising regenerative fibroblast-conditioned media. Regenerative fibroblast cells can be cultured under conditions sufficient to upregulate production of one or more growth factors in the regenerative fibroblast-conditioned media, and the regenerative fibroblast-conditioned media can be concentrated. The regenerative fibroblast-conditioned media can also be administered locally or systemically to the individual.

Also provided in one embodiment is a method of oocyte production comprising culturing isolated fibroblast cells in the presence of an agent that differentiates the fibroblast cells into an oocyte, thereby producing an oocyte. The agent can be selected from the group consisting of transforming growth factor, bone morphogenic protein, Wnt family protein, kit-ligand, leukemia inhibitory factor, meiosis-activating sterol, modulator of Id protein function, and modulator of Snail/Slug transcription factor function.

Provided in another embodiment is a method of oocyte production comprising administering fibroblast-derived germline cells to an individual, wherein the cells engraft into a tissue and differentiate into an oocyte, thereby producing an oocyte. The fibroblast-derived germline cells can be obtained by culturing fibroblasts with transforming growth factor, bone morphogenic protein, Wnt family protein, kit-ligand, leukemia inhibitory factor, meiosis-activating sterol, modulator of Id protein function, modulator of Snail/Slug transcription factor function, or a combination thereof. Further, the fibroblast-derived germline cells can be germline stem cells and/or germline progenitor cells.

Another embodiment provides a method of inducing folliculogenesis comprising administering fibroblast-derived germline cells to an ovary, wherein the cells engraft into the ovary and differentiate into an oocyte within a follicle. The fibroblast-derived germline cells can be germline stem cells and/or germline progenitor cells.

A further embodiment provides a pharmaceutical composition comprising a purified population of cells that are mitotically competent, have an XX karyotype and express Vasa, Dazl and Stella. The purified population of cells can be capable of differentiating into female germline cells. The female germline cells can be female germline progenitor cells and/or female germline stem cells. The cells can be purified from fibroblasts and can be mammalian or human cells. In some embodiments, the purified population of cells is about 50 to about 55%, about 55 to about 60%, about 65 to about 70%, about 70 to about 75%, about 75 to about 80%, about 80 to about 85%, about 85 to about 90%, about 90 to about 95% or about 95 to about 100% of the cells in the composition. The pharmaceutical composition may further comprise a pharmaceutically acceptable carrier. Administration of the composition can be local or systemic, and local administration can be inside the ovary, in the peri-ovary area, or a combination thereof.

It is specifically contemplated that any limitation discussed with respect to one embodiment of the disclosure may apply to any other embodiment of the disclosure.

Furthermore, any composition of the disclosure may be used in any method of the disclosure, and any method of the disclosure may be used to produce or to utilize any composition of the disclosure. Aspects of an embodiment set forth in the Examples are also embodiments that may be implemented in the context of embodiments discussed elsewhere in a different Example or elsewhere in the application, such as in the Brief Summary, Detailed Description, Claims, and Brief Description of the Drawings.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIG. 1 shows that intra-ovarian injection of fibroblasts into chemotherapy-treated mice reduces FSH production to levels similar to those observed in control, untreated mice. From left to right, the bars are Control, Chemo, Chemo+MSCs, and Chemo+Fibroblasts.

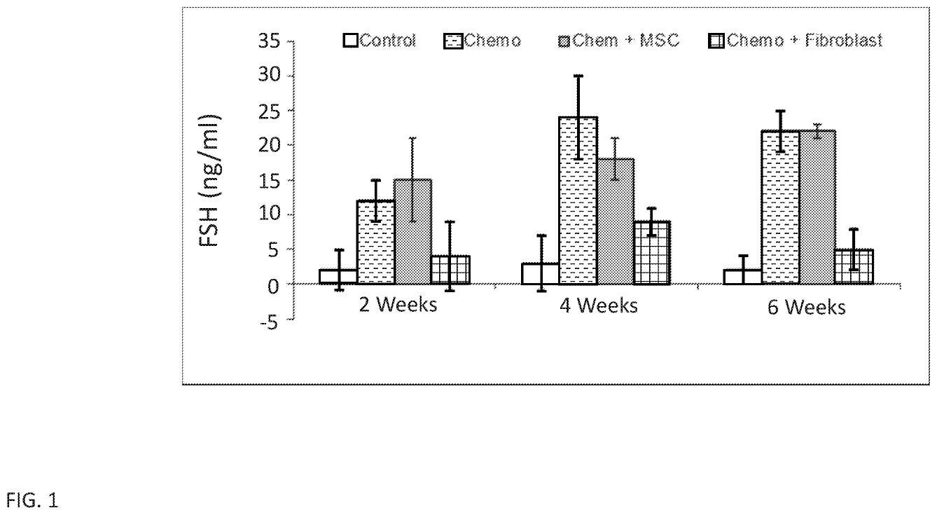

FIG. 2 shows that intravenous injection of fibroblasts into chemotherapy-treated mice reduces FSH production to levels similar to those observed in control, untreated mice. From left to right, the bars are Control, Chemo, Chemo+MSCs, and Chemo+Fibroblasts.

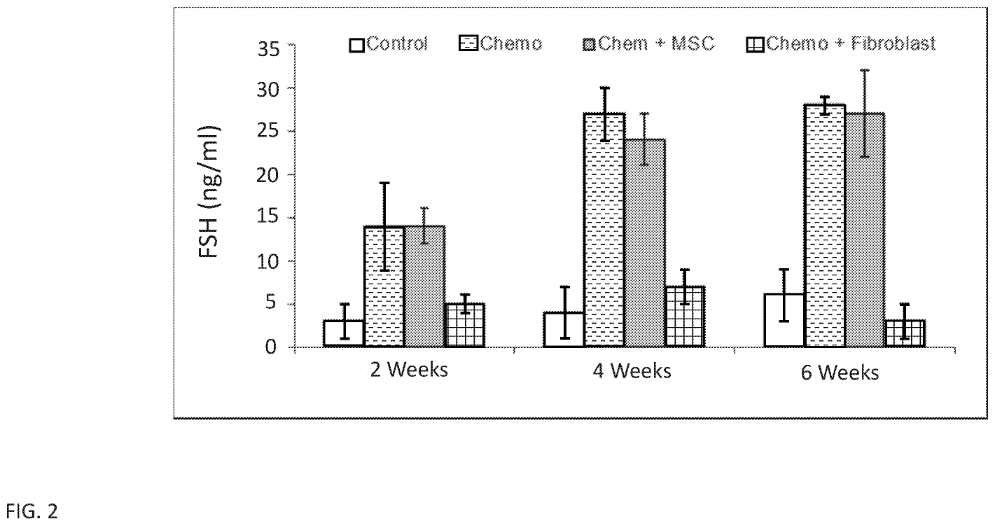

FIG. 3 shows that intra-ovarian injection of fibroblasts into chemotherapy-treated mice restores estradiol production to levels similar to those observed in control, untreated mice. From left to right, the bars are Control, Chemo, Chemo+MSCs, and Chemo+Fibroblasts.

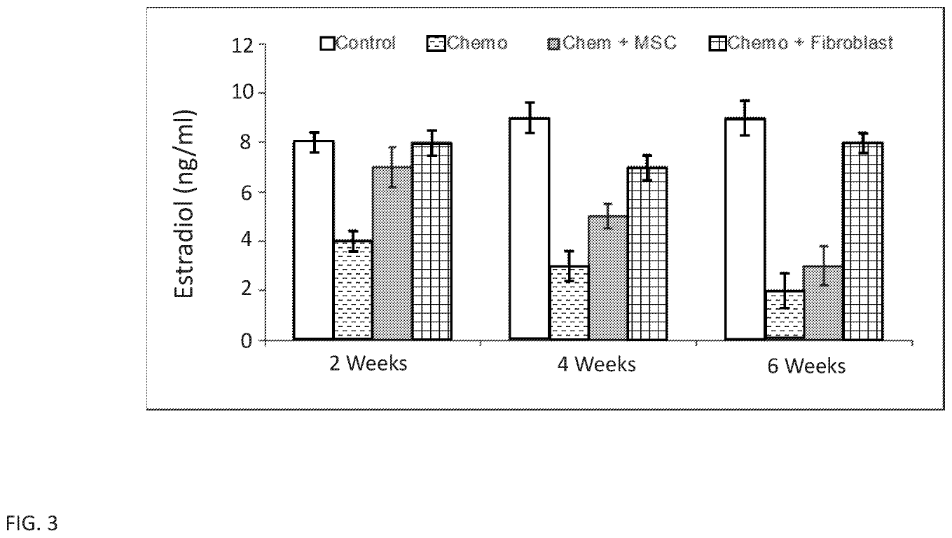

FIG. 4 shows that intravenous injection of fibroblasts into chemotherapy-treated mice restores estradiol production to levels similar to those observed in control, untreated mice. From left to right, the bars are Control, Chemo, Chemo+MSCs, and Chemo+Fibroblasts.

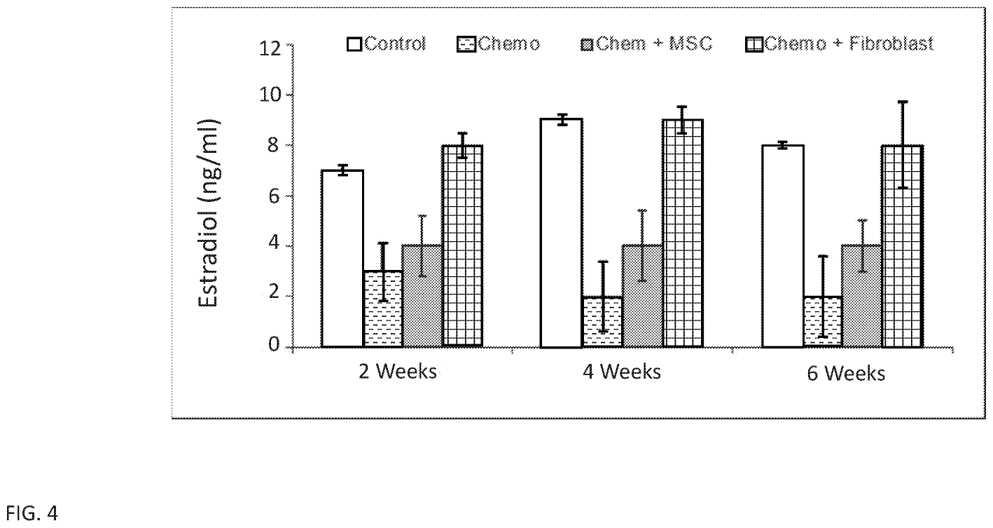

FIG. 5 shows that administration of fibroblasts into chemo-therapy treated mice increases the number of pregnancies compared to administration of bone marrow mesenchymal stem cells. From left to right, the bars are Control, Chemo, Chemo+MSCs, and Chemo+Fibroblasts.

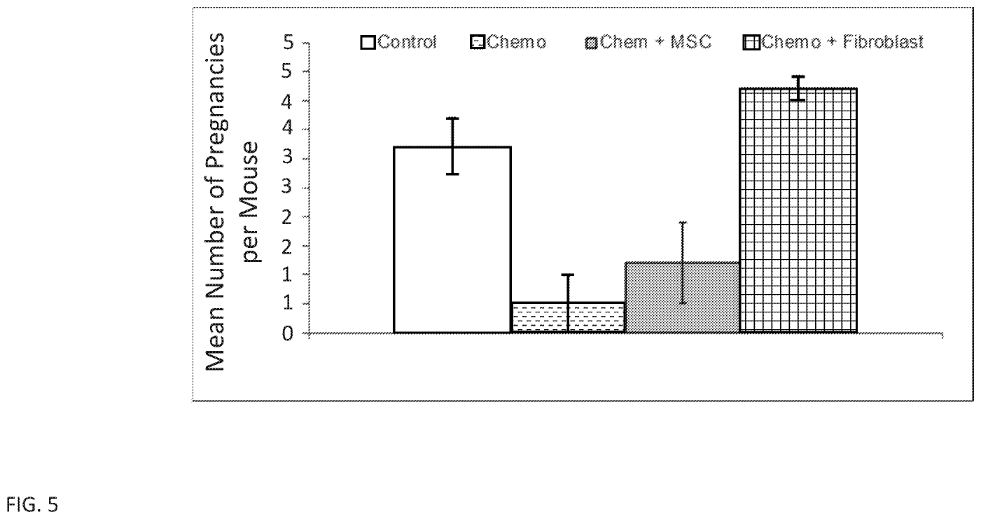

### DETAILED DESCRIPTION

### I. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

A variety of aspects of this disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. When ranges are present, the ranges may include the range endpoints.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

Furthermore, the particular features, structures, or characteristics may be combined in any suitable manner in one or more embodiments. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

As used herein, “allogeneic” refers to tissues or cells or other material from another body that in a natural setting are immunologically incompatible or capable of being immunologically incompatible, although from one or more individuals of the same species.

As used herein, “cell culture” means conditions wherein cells are obtained (e.g., from an organism) and grown under controlled conditions (“cultured” or grown “in culture”) outside of an organism. A primary cell culture is a culture of cells taken directly from an organism (e.g., tissue cells, blood cells, cancer cells, neuronal cells, fibroblasts, etc.). Cells are expanded in culture when placed in a growth medium under conditions that facilitate cell growth and/or division. The term “growth medium” means a medium sufficient for culturing cells. Various growth media may be used for the purposes of the present disclosure including, for example, Dulbecco's Modified Eagle Media (also known as Dulbecco's Minimal Essential Media) (DMEM), or DMEM-low glucose (also DMEM-LG herein). DMEM-low glucose may be supplemented with fetal bovine serum (e.g., about 10% v/v, about 15% v/v, about 20% v/v, etc.), antibiotics, antimycotics (e.g., penicillin, streptomycin, and/or amphotericin B), and/or 2-mercaptoethanol. Other growth media and supplementations to growth media are capable of being varied by the skilled artisan. The term “standard growth conditions” refers to culturing cells at 37° C. in a standard humidified atmosphere comprising 5% CO₂. While such conditions are useful for culturing, it is to be understood that such conditions are capable of being varied by the skilled artisan who will appreciate the options available in the art for culturing cells. When cells are expanded in culture, the rate of cell proliferation is sometimes measured by the amount of time needed for the cells to double in number. This is referred to as doubling time.

As used herein, “cell line” refers to a population of cells formed by one or more subcultivations of a primary cell culture. Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but not limited to seeding density, substrate, medium, growth conditions, and time between passaging.

As used herein, “conditioned medium” describes medium in which a specific cell or population of cells has been cultured for a period of time, and then removed, thus separating the medium from the cell or cells. When cells are cultured in a medium, they may secrete cellular factors that can provide trophic support to other cells. Such trophic factors include, but are not limited to hormones, cytokines, extracellular matrix (ECM), proteins, vesicles, antibodies, and granules. In this example, the medium containing the cellular factors is conditioned medium.

“Differentiation” (e.g., cell differentiation) describes a process by which an unspecialized (or “uncommitted”) or less specialized cell acquires the features (e.g., gene expression, cell morphology, etc.) of a specialized cell, such as a nerve cell or a muscle cell for example. A differentiated cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. In some embodiments of the disclosure, “differentiation” of fibroblasts to other cell types is described. This process may also be referred to as “transdifferentiation”.

As used herein, “dedifferentiation” refers to the process by which a cell reverts to a less specialized (or less committed) position within the lineage of a cell. As used herein, the lineage of a cell defines the heredity of the cell. The lineage of a cell places the cell within a hereditary scheme of development and differentiation. Within the context of the current disclosure, “dedifferentiation” may refer to fibroblasts acquiring more “immature” associated markers such as OCT4, NANOG, CTCFL, ras, raf, SIRT2 and SOX2. Additionally, “dedifferentiation” may mean acquisition of functional properties such as enhanced proliferation activity and/or migration activity towards a chemotactic gradient. In some embodiments fibroblasts may be “dedifferentiated” by treatment with various conditions, subsequent to which they are “differentiated” into other cell types.

“Fibroblasts” refer to a cell, progenitor cell, or differentiated cell and include isolated fibroblast cells or population(s) thereof capable of proliferating and differentiating into ectoderm, mesoderm, or endoderm. In some embodiments, fibroblasts are utilized in an autologous, allogenic, or xenogenic manner. As used herein, placental and adult-derived cellular populations are included in the definition of “fibroblasts.” In the context of the present disclosure, fibroblast cells may be derived through means known in the art from sources including at least foreskin, ear lobe skin, bone marrow, cord blood, placenta, amnion, amniotic fluid, umbilical cord, embryos, intraventricular cells from the cerebral spinal fluid, circulating fibroblast cells, mesenchymal stem cell associated cells, germinal cells, adipose tissue, exfoliated tooth-derived fibroblasts, hair follicle, dermis, skin biopsy, nail matrix, parthenogenically-derived fibroblasts, fibroblasts that have been reprogrammed to a dedifferentiated state, side population-derived fibroblasts, fibroblasts from plastic surgery-related by-products, and the like.

The term “fibroblast-derived product” (also “fibroblast-associated product” or “fibroblast-produced therapeutic factor”), as used herein, refers to a molecular or cellular agent derived or obtained from one or more fibroblasts. In some cases, a fibroblast-derived product is a molecular agent. Examples of molecular fibroblast-derived products include conditioned media from fibroblast culture, microvesicles obtained from fibroblasts, exosomes obtained from fibroblasts, apoptotic vesicles obtained from fibroblasts, nucleic acids (e.g., DNA, RNA, mRNA, miRNA, etc.) obtained from fibroblasts, proteins (e.g., growth factors, cytokines, etc.) obtained from fibroblasts, and lipids obtained from fibroblasts. In some cases, a fibroblast-derived product is a cellular agent. Examples of cellular fibroblast-derived products include cells (e.g., stem cells, hematopoietic cells, neural cells, etc.) produced by differentiation and/or de-differentiation of fibroblasts.

As used herein, the terms “germ cells” or “gamete-like cells” describes cells having characteristics of primordial germ cells, spermatocytes, oocytes, and the like. As used here, the term “oocyte” is used to describe the mature animal ovum, which is the final product of oogenesis. As used herein, the phrase “oocyte-like cell” broadly refers to any cell having characteristics of an oocyte or a precursor form of an oocyte, i.e., an oogonium, a primary oocyte or a secondary oocyte.

The term “ovarian failure,” as used herein, refers to both degeneration of the ovaries as a subject ages and primary or secondary ovary failure. Primary ovarian failure means that the ovaries do not function normally. This may be because they have been removed by surgery, or it may be caused by some cancer treatments and certain diseases or genetic conditions. In secondary ovarian failure, the ovaries are normal but there is a problem getting hormone signals to them from the brain. This is usually caused by diseases of the pituitary gland or hypothalamus.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

As used herein, “regenerative activities” include but are not limited to therapeutic functions, stimulation of angiogenesis, inhibition of inflammation, augmentation of tissue self-renewal, and/or preservation and/or stimulation of new oocytes, for example in part through activation of endogenous and/or exogenous stem and/or progenitor cells. Regenerative activities include the promotion of angiogenesis, suppression of inflammation, and secretion of growth factors such as IGF-1, EGF-1, FGF-2, VEGF, and FGF-11. Fibroblasts having regenerative activities can be isolated for specific markers and subsequently transfected with genes capable of endowing various therapeutic functions. Genes useful for stimulation of regenerative activities including augmentation of hematopoietic activity include interleukin-12 and interleukin-23 to stimulate proliferation of hematopoietic stem cells, for example. Other useful genes include interleukin-35, wherein interleukin-35 transfection allows for generation of cells possessing anti-inflammatory and angiogenic T regulatory cell activity, said cells possessing T regulatory cell activities include cells expressing the transcription factor FoxP3, as an example.

The term “subject,” as used herein, may be used interchangeably with the term “individual” and generally refers to an individual in need of a therapy. The subject can be a mammal, such as a human, dog, cat, horse, pig or rodent. The subject can be a patient, e.g., have or be suspected of having or at risk for having a disease or medical condition related to bone. For subjects having or suspected of having a medical condition directly or indirectly associated with bone, the medical condition may be of one or more types. The subject may have a disease or be suspected of having the disease. The subject may be asymptomatic. The subject may be of any gender. The subject may be of a certain age, such as at least 30, 35, 40, 45, 50, 55, 60, 65, 70, 75, 80, 85, 90, 95, or 100 or more.

As used herein, the phrase “subject in need thereof” or “individual in need thereof” refers to a subject or individual, as described infra, that suffers or is at a risk of suffering (e.g, pre-disposed such as genetically pre-disposed, or subjected to environmental conditions that pre-dispose, etc.) from the diseases or conditions listed herein (e.g, ovarian failure).

As used herein, the term “therapeutically effective amount” is synonymous with “effective amount”, “therapeutically effective dose”, and/or “effective dose” refers to an amount of an agent sufficient to produce a desired result or exert a desired influence on the particular condition being treated. In some embodiments, a therapeutically effective amount is an amount sufficient to ameliorate at least one symptom, behavior or event, associated with a pathological, abnormal or otherwise undesirable condition, or an amount sufficient to prevent or lessen the probability that such a condition will occur or re-occur, or an amount sufficient to delay worsening of such a condition. Effective amount can also mean the amount of a compound, material, or composition comprising a compound of the present disclosure that is effective for producing some desired effect, e.g., treating or preventing ovarian failure. For instance, in some embodiments, the effective amount refers to the amount of fibroblasts and/or fibroblast-conditioned media that can promote oocyte production, induce folliculogenesis, and/or treat or prevent ovarian failure in animals and humans. The effective amount may vary depending on the organism or individual treated.

The appropriate effective amount to be administered for a particular application of the disclosed methods can be determined by those skilled in the art, using the guidance provided herein. For example, an effective amount can be determined experimentally using various techniques and/or extrapolated from in vitro and in vivo assays including dose escalation studies. Various concentrations of an agent may be used in preparing compositions incorporating the agent to provide for variations in the age of the patient to be treated, the severity of the condition, and/or the duration of the treatment and the mode of administration. One skilled in the art will recognize that the condition of the individual can be monitored throughout the course of therapy and that the effective amount of a compound or composition disclosed herein that is administered can be adjusted accordingly. Further, one of skill in the art recognizes that an amount may be considered effective even if the medical condition is not totally eradicated but improved partially. For example, the medical condition may be halted or reduced or its onset delayed, a side effect from the medical condition may be partially reduced or completed eliminated, and so forth.

As used herein, the terms “treatment,” “treat,” or “treating” refers to intervention in an attempt to alter the natural course of the individual or cell being treated, and may be performed either for prophylaxis or during the course of pathology of a disease or condition, such as for example ovarian failure. Treatment may serve to accomplish one or more of various desired outcomes, including, for example, preventing occurrence or recurrence of disease, alleviation of symptoms, and diminishment of any direct or indirect pathological consequences of the disease, preventing disease spread, lowering the rate of disease progression, amelioration or palliation of the disease state, and remission or improved prognosis, and/or producing some desired effect, e.g. promotion of oocyte production and/or induction of folliculogenesis.

As used herein, a “trophic factor” describes a substance that promotes and/or supports survival, growth, proliferation and/or maturation of a cell. Alternatively or in addition, a trophic factor stimulates increased activity of a cell. The interaction between cells via trophic factors may occur between cells of different types. Cell interaction by way of trophic factors is found in essentially all cell types, and is a particularly significant means of communication among various types of cell types. Trophic factors also can function in an autocrine fashion, i.e., a cell may produce trophic factors that affect its own survival, growth, differentiation, proliferation, and/or maturation. Factors produced by fibroblasts are selected from HGF, FGF-1. FGF-2, FGF-5, CTNF, PDGF-BB, EGF, and/or TGF-fβ. Growth factors include one or more of GM-CSF, IL-15, IL-1a, IL-2, IL-4, IL-5, IL-6, IL-7, IL-8, MCP-1, TNFα, FGF-2, Flt-3, PDGF-AA, PDGF-BB, TGF-β1, TGF-β2, and TGF-β3.

### II. Generation of Regenerative Fibroblasts

Aspects of the present disclosure comprise cells useful in therapeutic methods and compositions. Cells disclosed herein include, for example, fibroblasts, stem cells (e.g., hematopoietic stem cells or mesenchymal stem cells), and endothelial progenitor cells. Cells of a given type (e.g., fibroblasts) may be used alone or in combination with cells of other types. For example, fibroblasts may be isolated and provided to a subject alone or in combination with one or more stem cells.

Certain further aspects of the present disclosure relate to the generation of fibroblasts and regenerative fibroblast cells for treating or preventing ovarian failure in individuals. Regenerative fibroblast cells may be generated by culturing fibroblasts under sufficient conditions to generate a regenerative fibroblast cell. In some embodiments, the fibroblast cells can provide a tissue with regenerative activity. In some embodiments, the method includes culturing the population of fibroblast regenerative cells under conditions that support proliferation of the cells. In additional embodiments, the fibroblast cells may be cultured under conditions that form tissue aggregate bodies. In some embodiments, the fibroblast cells are used to create other cell types for tissue repair or regeneration. Generation of fibroblasts has been described previously in the art and is incorporated herein. Generally, fibroblasts are harvested, for example, biopsied, from dissociated tissues of interest. Accomplishing the dissociation process can be performed mechanically or by treatment of tissue with enzymes, such as dispase or collagenase, which releases cells from the tissues. Subsequently, single cell fibroblasts are isolated based on plastic adherence and then allowed to expand and proliferate in liquid media.

Specific desirable properties of fibroblast cells of the present disclosure are the ability to suppress inflammation; differentiate into functional tissue; and/or induce local resident stem and/or progenitor cells to proliferate through secretion of soluble factors or membrane bound activities. Also desirable is the ability of fibroblast cells of the present disclosure to stimulate maturation of oocytes from oocyte progenitor cells and induce folliculogenesis. In one embodiment, fibroblast cells are collected from an autologous patient, expanded ex vivo, and reintroduced into the patient at a concentration and frequency sufficient to cause therapeutic benefit. The fibroblast cells are selected for the ability to cause suppress inflammation, regenerate functional tissue, induce proliferation, stimulate maturation of oocytes from oocyte progenitor cells, and/or induce folliculogenesis.

When selecting fibroblast cells, several factors must be taken into consideration, including the ability for ex vivo expansion without loss of therapeutic activity, ease of extraction, general potency of activity, and potential for adverse effects. Ex vivo expansion ability of fibroblasts can be measured using typical proliferation and colony assays known to one skilled in the art, while identification of therapeutic activity depends on functional assays that test biological activities correlated with therapeutic goals.

In some embodiments, assessment of therapeutic activity is performed using surrogate assays which detect one or more markers associated with a specific therapeutic activity. The markers detected can include but are not limited to DAZL, CD105, CD90, or a combination thereof. In some embodiments, assays used to identify therapeutic activity of fibroblast cell populations include evaluation of the production of one or more factors associated with desired therapeutic activity. In some embodiments, evaluation of the production of one or more factors to approximate therapeutic activity in vivo includes identification and quantification of the production of EGF, HGF, IGF, or a combination thereof.

In one embodiment, fibroblast cells are collected from an autologous patient, expanded ex vivo, and reintroduced into the patient at a concentration and frequency sufficient to cause therapeutic benefit. In other cases, the expanded cells are allogeneic or xenogenic with respect to a recipient individual. In some embodiments, fibroblasts of the present disclosure are used as precursor cells that differentiate following introduction into an individual. In some embodiments, fibroblasts are subjected to differentiation into a different cell type (e.g., a hematopoietic cell) prior to introduction into the individual.

Compositions of the present disclosure may be obtained from isolated fibroblast cells or a population thereof capable of proliferating and differentiating into ectoderm, mesoderm, or endoderm. In some embodiments, the fibroblasts possess the ability to differentiate to osteogenic, chondrogenic, and adipogenic lineage cells, as well as female germline cells. In some embodiments, the enriched population of fibroblast cells are about 6-12, 6-11, 6-10, 6-9, 6-8, 6-7, 7-12, 7-11, 7-10, 7-9, 7-8, 8-12, 8-11, 8-10, 8-9, 9-12, 9-11, 9-10, 10-12, 10-11, or 11-12 micrometers in size. The fibroblasts may be plastic adherent.

In some embodiments, the fibroblast cells are expanded in culture using one or more cytokines, chemokines and/or growth factors prior to administration to an individual in need thereof. The agent capable of inducing fibroblast expansion can be selected from the group consisting of TPO, SCF, IL-1, IL-3, IL-7, flt-3L, G-CSF, GM-CSF, Epo, FGF-1, FGF-2, FGF-4, FGF-20, VEGF, activin-A, IGF, EGF, NGF, LIF, PDGF, a member of the bone morphogenic protein family, and a combination thereof. The agent capable of inducing fibroblast differentiation can be selected from the group consisting of HGF, BDNF, VEGF, FGF1, FGF2, FGF4, FGF20, and a combination thereof.

In some embodiments, the fibroblast cells express proteins characteristic of normal fibroblasts including the fibroblast-specific marker, CD90 (Thy-1), a 35 kDa cell-surface glycoprotein, and the extracellular matrix protein, collagen. In some embodiments, an isolated fibroblast cell expresses at least one of Oct-4, Nanog, Sox-2, KLF4, c-Myc, Rex-1, GDF-3, LIF receptor, CD105, CD117, CD344, or Stella markers. In some embodiments, the fibroblasts express CD73, CD90, and/or CD105. In some embodiments, fibroblast cells are isolated and expanded and possess one or more markers selected from a group consisting of CD10, CD13, CD44, CD73, CD90, CD141, PDGFr-alpha, HLA-A, HLA-B, HLA-C, or a combination thereof. In some embodiments, fibroblasts of the present disclosure express telomerase, Nanog, Sox2, β-III-Tubulin, NF-M, MAP2, APP, GLUT, NCAM, NeuroD, Nurr1, GFAP, NG2, Olig1, Alkaline Phosphatase, Vimentin, Osteonectin, Osteoprotegrin, Osterix, Adipsin, Erythropoietin, SM22-α, HGF, c-MET, α-1-Antriptrypsin, Ceruloplasmin, AFP, PEPCK 1, BDNF, NT-4/5, TrkA, BMP2, BMP4, FGF2, FGF4, PDGF, PGF, TGFα, TGFβ, and/or VEGF. In some embodiments, the fibroblast cells do not produce one or more of CD14, CD31, CD34, CD45, CD117, CD141, HLA-DR, HLA-DP, HLA-DQ, or a combination thereof. In further embodiments, the fibroblast regenerative cell has enhanced expression of GDF-11 as compared to a control. In still further embodiments, the fibroblast cells express CD73, which is indicative of fibroblast cells having regenerative activity.

In some embodiments, an isolated fibroblast cell does not express at least one of MHC class I, MHC class II, CD45, CD13, CD49c, CD66b, CD73, CD105, or CD90 cell surface proteins. Such isolated fibroblast cells may be used as a source of conditioned media. In some embodiments, the method optionally includes the step of depleting cells expressing stem cell surface markers or MHC proteins from the cell population, thereby isolating a population of stem cells. In some embodiments, the cells to be depleted express MHC class I, CD66b, glycophorin a, or glycophorin b.

In some cases, fibroblast cells are obtained from a biopsy, and the donor providing the biopsy may be either the individual to be treated (autologous), or the donor may be different from the individual to be treated (allogeneic). In some embodiments, the fibroblast cells are xenogenic with respect to a recipient individual. In some embodiments wherein allogeneic fibroblast cells are utilized for an individual, the fibroblast cells may come from one or a plurality of donors. In some embodiments fibroblasts are used from young, pre-pubescent donors. In some embodiments, steps are taken to protect allogeneic or xenogenic cells from immune-mediated rejection by the recipient. Steps include encapsulation, co-administration of an immune suppressive agent, transfection of said cells with immune suppressory agent, or a combination thereof. In some embodiments, tolerance to the cells is induced through immunological means. In some embodiments, fibroblasts are transfected with genes to allow for enhanced growth and to overcome the Hayflick limit. Subsequent to derivation by biopsy, the fibroblasts are expanded in culture using standard cell culture techniques.

Biopsy is performed by extracting tissue, usually 0.1 grams to 10 grams. In some situations biopsy may be performed using a biopsy gun or alternatively using a scalpel. In one embodiment, biopsies are from skin tissue (dermis and epidermis layers) from a subject's post-auricular area. In one embodiment, the starting material is composed of three 3-mm punch skin biopsies collected using standard aseptic practices, though the methods disclosed herein for preparing a skin biopsy tissue would apply equally to biopsies of other tissue types. The biopsies are collected by the treating physician, placed into a vial containing sterile phosphate buffered saline (PBS), and stored at 2-8° C. Upon initiation of the process, biopsies are inspected, and accepted biopsy tissues are washed prior to enzymatic digestion. After washing, a Liberase Digestive Enzyme Solution is added without mincing, and the biopsy tissue is incubated at 37.0±2° C. for one hour. Time of biopsy tissue digestion is a critical process parameter that can affect the viability and growth rate of cells in culture. Liberase is a collagenase/neutral protease enzyme cocktail obtained formulated from Lonza Walkersville, Inc. (Walkersville, Md.) and unformulated from Roche Diagnostics Corp. (Indianapolis, Ind.). Other commercially available collagenases may also be used, such as Serva Collagenase NB6 (Helidelburg, Germany).

After digestion, Iscove's Complete Growth Media (IMDM, GA, 10% Fetal Bovine Serum (FBS)) is added to neutralize the enzyme, and cells are pelleted by centrifugation and re-suspended in 5.0 mL IMDM. Alternatively, full enzymatic inactivation is achieved by adding IMDM without centrifugation. Additional IMDM is added prior to seeding of the cell suspension into a particular flask (such as a T-175 cell culture flask) for initiation of cell growth and expansion. Alternatively, a T-75, T-150, T-185, or T-225 flask can be used in place of the T-175 flask. Cells are incubated at 37.0±2° C. with 5.0±1.0% CO₂ and supplemented with fresh IMDM every three to five days by removing half of the IMDM and replacing it with the same volume with fresh media. Alternatively, full IMDM replacements can be performed.

In specific cases, cells are not cultured in the T-175 flask for more than 30 days prior to passaging. Confluence is monitored throughout the process to ensure adequate seeding densities upon culture splitting. When cell confluence is greater than or equal to 40% in the T-175 flask, the cells are passaged by removing the spent media, washing the cells, and treating the cells with Trypsin-EDTA to release adherent cells in the flask into the solution. Cells are then trypsinized and seeded into a T-500 flask for continued cell expansion. Alternately, one or two T-300 flasks, One Layer Cell Stack (1 CS), One Layer Cell Factory (1 CF), or a Two Layer Cell Stack (2 CS) can be used in place of the T-500 Flask.

Morphology may be evaluated at each passage and prior to harvest to monitor culture purity throughout the process by comparing the observed sample with visual standards for morphological examination of cell cultures. Typical fibroblast morphologies when growing in cultured monolayers include elongated, fusiform, or spindle-shaped cells with slender extensions or larger, flattened stellate cells with cytoplasmic leading edges. A mixture of these morphologies may also be observed. Fibroblasts in less confluent areas can be similarly shaped but randomly oriented. The presence of keratinocytes in cell cultures may also be evaluated. Keratinocytes are round and irregularly shaped and, at higher confluence, appear to be organized in a cobblestone formation. At lower confluence, keratinocytes are observable in small colonies.

Cells are incubated at 37±2° C. with 5.0±1.0% CO₂ and passaged every three to five days for cells in T-500 flasks and every five to seven days for cells in ten layer cell stacks (10CS). Cells should not be cultured for more than 10 days prior to passaging. When cell confluence in a T-500 flask is ≥95%, cells are passaged to a 10 Layer Cell Stack (10 CS) culture vessel. Alternately, two Five Layer Cell Stacks (5 CS) or a 10 Layer Cell Factory (10 CF) can be used in place of the 10 CS. Passage to the 10 CS is performed by removing the spent media, washing the cells, and treating with Trypsin-EDTA to release adherent cells in the flask into the solution. Cells are then transferred to the 10 CS. Additional IMDM is added to neutralize the trypsin, and the cells from the T-500 flask are pipetted into a 2 L bottle containing fresh IMDM.

The contents of the 2 L bottle may be transferred into the 10 CS and seeded across all layers of the 10 CS. Cells are then incubated at 37.0±2° C. with 5.0±1.0% CO₂ and supplemented with fresh IMDM every five to seven days. In some cases, cells should not be cultured in the 10 CS for more than 20 days prior to passaging. In one embodiment, the passaged fibroblasts are rendered substantially free of immunogenic proteins present in the culture medium by incubating the expanded fibroblasts for a period of time in protein-free medium. When cell confluence in the 10 CS is ≥95%, cells are harvested. Harvesting is performed by removing the spent media, washing the cells, treating with Trypsin-EDTA to release adherent cells into the solution, and adding additional IMDM to neutralize the trypsin. Cells are collected by centrifugation and resuspended, and quality control testing is performed to determine total viable cell count and cell viability as well as sterility and the presence of endotoxins.

In some embodiments, the fibroblast dosage formulation is a suspension of fibroblasts obtained from a biopsy using standard tissue culture procedures. The cells in the formulation display typical fibroblast morphologies when growing in cultured monolayers. Specifically, cells may display an elongated, fusiform or spindle appearance with slender extensions, or cells may appear as larger, flattened stellate cells which may have cytoplasmic leading edges. A mixture of these morphologies may also be observed. The cells may also express proteins characteristic of normal fibroblasts, including the fibroblast-specific marker CD90 (Thy-1), a 35 kDa cell-surface glycoprotein, and the extracellular matrix protein collagen.

In one embodiment, regenerative fibroblast cells are purified from cord blood. Cord blood fibroblast cells are fractionated, and the fraction with enhanced therapeutic activity is administered to the patient. In some embodiments, cells with therapeutic activity are enriched based on physical differences (e.g., size and weight), electrical potential differences (e.g., charge on the membrane), differences in uptake or excretion of certain compounds (e.g., rhodamine-123 efflux), as well as differences in expression marker proteins (e.g., CD73). Distinct physical property differences between stem cells with high proliferative potential and low proliferative potential are known. Accordingly, in some embodiments, cord blood fibroblast cells with a higher proliferative ability are selected, whereas in other embodiments, a lower proliferative ability is desired. In some embodiments, cells are directly injected into the area of need and should be substantially differentiated. In other embodiments, cells are administered systemically and should be less differentiated, so as to still possess homing activity to the area of need.

In embodiments where specific cellular physical properties are the basis of differentiating between cord blood fibroblast cells with various biological activities, discrimination on the basis of physical properties can be performed using a Fluorescent Activated Cell Sorter (FACS), through manipulation of the forward scatter and side scatter settings. Other embodiments include methods of separating cells based on physical properties using filters with specific size ranges, density gradients, and pheresis techniques. In embodiments where differentiation is based on electrical properties of cells, techniques such as electrophotoluminescence are used in combination with a cell sorting means such as FACS. In some embodiments, selection of cells is based on ability to uptake certain compounds as measured by the ALDESORT system, which provides a fluorescent-based means of purifying cells with high aldehyde dehydrogenase activity. Without being bound by theory, cells with high levels of this enzyme are known to possess higher proliferative and self-renewal activities in comparison to cells possessing lower levels. Further embodiments include methods of identifying cells with high proliferative activity by identifying cells with ability to selectively efflux certain dyes such as rhodamine-123, Hoechst 33342, or a combination thereof. Without being bound to theory, cells possessing this property often express the multidrug resistance transport protein ABCG2 and are known for enhanced regenerative ability compared to cells which do not possess this efflux mechanism.

In some embodiments, cord blood cells are purified for certain therapeutic properties based on the expression of markers. In one particular embodiment, cord blood fibroblast are purified for cells with the endothelial precursor cell phenotype. Endothelial precursor cells or progenitor cells expressing markers such as but not limited to CD133, CD34, or a combination thereof and/or fibroblasts expressing markers such as but not limited to CD90, CD73, CD105 and HLA-G may be purified by positive or negative selection using techniques such as magnetic activated cell sorting (MACS), affinity columns, FACS, panning, other means known in the art, or a combination thereof. In some embodiments, cord blood-derived endothelial progenitor cells are administered directly into the target tissue, while in other embodiments, the cells are administered systemically. In some embodiments, the endothelial precursor cells are differentiated in vitro and infused into a patient. Verification of endothelial differentiation is performed by assessing ability of cells to bind FITC-labeled Ulex europaeus agglutinin-1, ability to endocytose acetylated Di-LDL, and the expression of endothelial cell markers such as PECAM-1, VEGFR-2, or CD31.

In some embodiments, cord blood fibroblast cells are endowed with desired activities prior to administration into the patient. In one specific embodiment, cord blood cells are “activated” ex vivo by brief culture in hypoxic conditions to upregulate nuclear translocation of the HIF-1α transcription factor and endow the cord blood cells with enhanced angiogenic potential. In some embodiments, hypoxia is achieved by culture of cells in conditions of 0.1% oxygen to 10% oxygen. In further embodiments, hypoxia is achieved by culture of cells in conditions of 0.5% oxygen and 5% oxygen. In further embodiments, hypoxia is achieved by culture of cells in conditions of about 1% oxygen. Cells may be cultured for a variety of time points ranging from 1 hour to 72 hours in some embodiments, to 13 hours to 59 hours in further embodiments and around 48 hours in still further embodiments. In one embodiment, cord blood cells are assessed for angiogenic or other desired activities prior to administration of the cord blood cells into the patient. Assessment methods are known in the art and include measurement of angiogenic factors, the ability to support cell viability and activity, and the ability to induce regeneration of the cellular components.

In additional embodiments, cord blood fibroblast cells are endowed with additional therapeutic properties through treatment ex vivo with factors such as de-differentiating compounds, proliferation-inducing compounds, compounds known to endow and/or enhance cord blood cells with useful properties, or a combination thereof. In one embodiment, cord blood cells are cultured with an inhibitor of the enzyme GSK-3 to enhance expansion of cells with pluripotent characteristics while maintaining the rate of differentiation. In another embodiment, cord blood cells are cultured in the presence of a DNA methyltransferase inhibitor such as 5-azacytidine to confer a “de-differentiation” effect. In another embodiment cord blood fibroblast cells are cultured in the presence of a differentiation agent that induces the cord blood stem cells to generate enhanced numbers of cells useful for treatment after the cord blood cells are administered to a patient.

In one embodiment, regenerative fibroblasts are purified from placental tissues. In contrast to cord blood fibroblast cells, in some embodiments, placental fibroblast cells are purified directly from placental tissues including the chorion, amnion, and villous stroma. In another embodiment, placental tissue is mechanically degraded in a sterile manner and treated with enzymes to allow dissociation of the cells from the extracellular matrix. Such enzymes include but are not restricted to trypsin, chymotrypsin, collagenases, elastase, hyaluronidase, or a combination thereof. In some embodiments, placental cell suspensions are subsequently washed, assessed for viability, and used directly by administration locally or systemically. In some embodiments, placental cell suspensions are purified to obtain certain populations with increased biological activity.

Purification may be performed using means known in the art including those used for purification of cord blood fibroblast cells. In some embodiments, purification may be achieved by positive selection for cell markers including SSEA3, SSEA4, TRA1-60, TRA1-81, c-kit, and Thy-1. In some embodiments, cells are expanded before introduction into the human body. Expansion can be performed by culture ex vivo with specific growth factors. Embodiments described for cord blood and embryonic stem cells also apply to placental stem cells.

Fibroblasts can also be extracted from various tissues including but not limited to umbilical cord, skin, adipose tissue, bone marrow, cord blood, and omental tissue. In some embodiments, fibroblasts are obtained from a source selected from the group comprising dermal fibroblasts; placental fibroblasts; omental tissue fibroblasts; adipose fibroblasts; bone marrow fibroblasts; foreskin fibroblasts; umbilical cord fibroblasts; cord blood fibroblasts; amniotic fluid; embryonic fibroblasts; hair follicle-derived fibroblasts; nail-derived fibroblasts; endometrial-derived fibroblasts; keloid-derived fibroblasts; ear lobe skin; plastic surgery-related by-products; or a combination thereof. In some embodiments, fibroblasts are fibroblasts isolated from skin, placenta, omentum, adipose tissue, bone marrow, foreskin, umbilical cord, cord blood, amnion, embryos, hair follicle, nails, endometrium, keloids, ear lobe, Wharton's Jelly, and/or plastic surgery-related by-products.

In some embodiments, the fibroblasts are fibroblasts isolated from peripheral blood of a subject who has been exposed to conditions sufficient to stimulate fibroblasts from the subject to enter the peripheral blood. In another embodiment, fibroblast cells are mobilized by use of a mobilizing agent or therapy for treatment of ovarian failure. In some embodiments, the conditions and/or agents sufficient to stimulate fibroblasts from the subject to enter the peripheral blood comprise administration of G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA reductase inhibitors, small molecule antagonists of SDF-1, or a combination thereof. In some embodiments, the mobilization therapy is selected from a group comprising exercise, hyperbaric oxygen, autohemotherapy by ex vivo ozonation of peripheral blood, induction of SDF-1 secretion in an anatomical area outside of the bone marrow, or a combination thereof. In some embodiments, the committed fibroblasts can express the marker CD133 or CD34 and are mobilized.

Any of the fibroblast cell populations disclosed herein may be used as a source of conditioned media and/or exosomes, apoptotic bodies, or microvesicles produced by fibroblasts. The cells may be cultured alone, or may by cultured in the presence of other cells in order to further upregulate production of growth factors in the conditioned media. In some embodiments, fibroblasts of the present disclosure are cultured with an inhibitor of mRNA degradation. In some embodiments, fibroblasts are cultured under conditions suitable to support differentiation and/or reprogramming of the fibroblasts. In some embodiments, such conditions comprise temperature conditions of between 30° C. and 38° C., between 31° C. and 37° C., or between 32° C. and 36° C. In some embodiments, such conditions comprise glucose at or below 4.6 g/l, 4.5 g/l, 4 g/l, 3 g/l, 2 g/l or 1 g/l. In some embodiments, such conditions comprise glucose of about 1 g/l.

Various terms are used to describe cells in culture. Cell culture refers generally to cells taken from a living organism and grown under controlled condition (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number, or the “doubling time.” Fibroblast cells used in the disclosed methods can undergo at least 25, 30, 35, or 40 doublings prior to reaching a senescent state. Methods for deriving cells capable of doubling to reach 10¹⁴ cells or more are provided. In some embodiments, methods are used to derive cells that can double sufficiently to produce at least about 10¹⁴, 10¹⁵, 10¹⁶, or 10¹⁷ or more cells when seeded at from about 10³ to about 10⁶ cells/cm² in culture within 80, 70, or 60 days or less. In some embodiments, fibroblasts are transfected with one or more genes to allow for enhanced growth and overcoming of the Hayflick limit.

When referring to cultured cells, including fibroblast cells and vertebrae cells, the term senescence (also “replicative senescence” or “cellular senescence”) refers to a property attributable to finite cell cultures; namely, their inability to grow beyond a finite number of population doublings (sometimes referred to as Hayflick's limit). Although cellular senescence was first described using fibroblast-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone. Thus, cells made quiescent by removing essential growth factors are able to resume growth and division when the growth factors are re-introduced, and thereafter carry out the same number of doublings as equivalent cells grown continuously. Similarly, when cells are frozen in liquid nitrogen after various numbers of population doublings and then thawed and cultured, they undergo substantially the same number of doublings as cells maintained unfrozen in culture. Senescent cells are not dead or dying cells; they are resistant to programmed cell death (apoptosis) and can be maintained in their nondividing state for as long as three years. These cells are alive and metabolically active, but they do not divide.

In some embodiments, the present disclosure utilizes exosomes derived from fibroblasts as a therapeutic modality. Exosomes derived from fibroblasts may be used in addition to, or in place of, fibroblasts in the various methods and compositions disclosed herein. Exosomes, also referred to as “microparticles” or “particles,” may comprise vesicles or a flattened sphere limited by a lipid bilayer. The microparticles may comprise diameters of 40-100 nm. The microparticles may be formed by inward budding of the endosomal membrane. The microparticles may have a density of about 1.13-1.19 g/ml and may float on sucrose gradients. The microparticles may be enriched in cholesterol and sphingomyelin, and lipid raft markers such as GM1, GM3, flotillin and the src protein kinase Lyn. The microparticles may comprise one or more proteins present in fibroblast, such as a protein characteristic or specific to the fibroblasts or fibroblast conditioned media. They may comprise RNA, for example miRNA. The microparticles may possess one or more genes or gene products found in fibroblasts or medium which is conditioned by culture of fibroblasts. The microparticles may comprise molecules secreted by the fibroblasts. Such a microparticle, and combinations of any of the molecules comprised therein, including in particular proteins or polypeptides, may be used to supplement the activity of, or in place of, the fibroblasts for the purpose of, for example, treating or preventing ovarian failure. The microparticle may comprise a cytosolic protein found in cytoskeleton e.g., tubulin, actin and actin-binding proteins, intracellular membrane fusions and transport, e.g., annexins and rab proteins, signal transduction proteins, e.g., protein kinases, 14-3-3 and heterotrimeric G proteins, metabolic enzymes, e.g., peroxidases, pyruvate and lipid kinases, and enolase-1 and the family of tetraspanins, e.g., CD9, CD63, CD81 and CD82. In particular, the microparticle may comprise one or more tetraspanins.

As disclosed herein, fibroblasts may secrete one or more factors prior to or following introduction into an individual. Such factors include, but are not limited to, growth factors, trophic factors and cytokines. In some instances, the secreted factors can have a therapeutic effect in the individual. In some embodiments, a secreted factor activates the same cell. In some embodiments, the secreted factor activates neighboring and/or distal endogenous cells. In some embodiments, the secreted factor stimulated cell proliferation and/or cell differentiation. In some embodiments, fibroblasts secrete a cytokine or growth factor selected from human growth factor, fibroblast growth factor, nerve growth factor, insulin-like growth factors, hematopoietic stem cell growth factors, a member of the fibroblast growth factor family, a member of the platelet-derived growth factor family, a vascular or endothelial cell growth factor, and a member of the TGFβ family.

In some embodiments, fibroblasts are manipulated such that they do not produce one or more factors. Under appropriate conditions, fibroblasts may be capable of producing interleukin-1 (IL-1) and/or other inflammatory cytokines. In some embodiments, fibroblasts of the present disclosure are modified (e.g., by gene editing) to prevent or reduce expression of IL-1 or other inflammatory cytokines. For example, in some embodiments, fibroblasts are fibroblasts having a deleted or non-functional IL-1 gene, such that the fibroblasts are unable to express IL-1. Such modified fibroblasts may be useful in the therapeutic methods of the present disclosure by having limited pro-inflammatory capabilities when provided to a subject. In some embodiments, fibroblasts are treated with (e.g., cultured with) TNF-α, thereby inducing expression of growth factors and/or fibroblast proliferation.

In some embodiments, fibroblasts are transfected with one or more angiogenic genes to enhance ability to promote ovary function. An “angiogenic gene” describes a gene encoding for a protein or polypeptide capable of stimulating or enhancing angiogenesis in a culture system, tissue, or organism. Examples of angiogenic genes which may be useful in transfection of fibroblasts include activin A, adrenomedullin, aFGF, ALK1, ALK5, ANF, angiogenin, angiopoietin-1, angiopoietin-2, angiopoietin-3, angiopoietin-4, bFGF, B61, bFGF inducing activity, cadherins, CAM-RF, cGMP analogs, ChDI, CLAF, claudins, collagen, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TGF-beta, PD-ECGF, PDGF, IGF, IL8, growth hormone, fibrin fragment E, FGF-5, fibronectin, fibronectin receptor, Factor X, HB-EGF, HBNF, HGF, HUAF, heart derived inhibitor of vascular cell proliferation, Ill, IGF-2 IFN-gamma, α1β1 integrin, α2β1 integrin, K-FGF, LIF, leiomyoma-derived growth factor, MCP-1, macrophage-derived growth factor, monocyte-derived growth factor, MD-ECI, MECIF, MMP2, MMP3, MMP9, urokiase plasminogen activator, neuropilin, neurothelin, nitric oxide donors, nitric oxide synthases (NOSs), notch, occludins, zona occludins, oncostatin M, PDGF, PDGF-B, PDGF receptors, PDGFR-β3, PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-beta, Tie 1, Tie2, TGF-β3, TGF-β3 receptors, TIMPs, TNF-α, transferrin, thrombospondin, urokinase, VEGF-A, VEGF-B, VEGF-C, VEGF-D, VEGF-E, VEGF, VEGF(164), VEGI, and EG-VEGF. Fibroblasts transfected with one or more angiogenic factors may be used in the disclosed methods of disease treatment or prevention.

In some embodiments, fibroblasts are manipulated or stimulated to produce one or more factors. In some embodiments, fibroblasts are manipulated or stimulated to produce leukemia inhibitory factor (LIF), brain-derived neurotrophic factor (BDNF), epidermal growth factor receptor (EGF), basic fibroblast growth factor (bFGF), FGF-6, glial-derived neurotrophic factor (GDNF), granulocyte colony-stimulating factor (GCSF), hepatocyte growth factor (HGF), IFN-γ, insulin-like growth factor binding protein (IGFBP-2), IGFBP-6, IL-1ra, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-β), vascular endothelial growth factor (VEGF), VEGF-D, urokinase plasminogen activator receptor (uPAR), bone morphogenetic protein 4 (BMP4), IL1-a, IL-3, leptin, stem cell factor (SCF), stromal cell-derived factor-1 (SDF-1), platelet derived growth factor-BB (PDGFBB), transforming growth factors beta (TGFβ-1) and/or TGFβ-3. Factors from manipulated or stimulated fibroblasts may be present in conditioned media and collected for therapeutic use.

Conditions promoting certain types of fibroblast proliferation or differentiation can also be used during the culture of regenerative fibroblasts cells. These conditions include but are not limited to one or more of alteration in temperature, oxygen/carbon dioxide content, and/or turbidity of the growth media, and/or exposure to small molecule modifiers of cell culture like nutrients, certain enzyme inhibitors, certain enzyme stimulators, and/or histone deacetylase inhibitors, such as valproic acid.

In some embodiments, fibroblast cells are cultured under conditions to suppress expression of one or more apoptosis-associated genes. Anti-apoptosis genes allow for enhanced survival of fibroblasts in vitro and in vivo. In some embodiments, the one or more apoptosis-associated genes are selected from the group consisting of Fas, FasL, CASP1 (ICE), CASP10 (MCH4), CASP14, CASP2, CASP3, CASP4, CASP5, CASP6, CASP7, CASP8, CASP9, CFLAR (CASPER), CRADD, PYCARD (TMS1/ASC), ABL1, AKT1, BAD, BAK1, BAX, BCL2L11, BCLAF1, BID, BIK, BNIP3, BNIP3L, CASP1 (ICE), CASP10 (MCH4), CASP14, CASP2, CASP4, CASP6, CASP8, CD70 (TNFSF7), CIDEB, CRADD, FADD, FASLG (TNFSF6), HRK, LTA (TNFB), NOD1 (CARD4), PYCARD (TMS1/ASC), RIPK2, TNF, TNFRSF10A, TNFRSF10B (DR5), TNFRSF25 (DR3), TNFRSF9, TNFSF10 (TRAIL), TNFSF8, TP53, TP53BP2, TRADD, TRAF2, TRAF3, TRAF4, and a combination thereof.

In some embodiments, the conditions to suppress expression of apoptosis-associated genes comprise administration of an antisense oligonucleotide which activates RNAse H. In some embodiments, the conditions to suppress expression of apoptosis-associated genes comprise administration of an agent capable of inducing RNA interference, including short interfering RNA and/or short hairpin RNA.

In some embodiments, fibroblasts are cultured under hypoxic conditions prior to administration in order to confer enhanced cytokine production properties and stimulate migration toward chemotactic gradients. Without wishing to be bound theory, protocols to enhance the regenerative potential of non-fibroblast cells using hypoxia can be modified or adapted for use with fibroblasts. For example, in one study, short-term exposure of MSCs to 1% oxygen increased mRNA and protein expression of the chemokine receptors CX3CR1 and CXCR4. After 1-day exposure to low oxygen, in vitro migration of MSCs in response to the fractalkine and SDF-lalpha increased in a dose dependent manner, while blocking antibodies for the chemokine receptors significantly decreased migration. Xenotypic grafting of cells from hypoxic cultures into early chick embryos demonstrated more efficient grafting of cells from hypoxic cultures compared to cells from normoxic cultures, and cells from hypoxic cultures generated a variety of cell types in host tissues. Other descriptions of hypoxic conditioning are described in the art. For example, cells can be cultured in hypoxic conditions or with gases that displace oxygen and/or cells can be treated with hypoxic mimetics.

In some embodiments chemical agents such as iron chelators, for example, deferoxamine, are added during in vitro incubation or in vivo to enhance migration of fibroblasts to an area in need. Another useful preconditioning agent is all trans retinoic acid, used at concentrations similar to those described for MSC, for example, between 0.001 uM to 1 uM or between 0.01 μM and 1 μM.

Without wishing to be bound by theory, hypoxia has been demonstrated to induce expression of angiogenic genes in cells. For example, studies involving hypoxic preconditioning (HPC) of MSC exposed MSCs to 0.5% oxygen for 24, 48, or 72 h before evaluating the expression of prosurvival, proangiogenic, and functional markers, such as hypoxia-inducible factor-1α, VEGF, phosphorylated Akt, survivin, p21, cytochrome c, caspase-3, caspase-7, CXCR4, and c-Met. MSCs exposed to 24-h hypoxia showed reduced apoptosis and had significantly higher levels of prosurvival, proangiogenic, and prodifferentiation proteins compared to MSCs exposed to 72-h hypoxia. Cells taken directly from a cryopreserved state did not respond as effectively to 24-h HPC as those cells cultured under normoxia before HPC. Cells cultured under normoxia before HPC showed decreased apoptosis and enhanced expression of connexin-43, cardiac myosin heavy chain, and CD31. The preconditioned cells were also able to differentiate into cardiovascular lineages. The results of the study suggest that MSCs cultured under normoxia before 24-h HPC are in a state of optimal expression of prosurvival, proangiogenic, and functional proteins that may increase subsequent survival after engraftment of the cells. The same conditions used to culture MSCs can be used to culture the fibroblasts of the present disclosure.

Thus, in some embodiments, regenerative fibroblast cells are exposed to 0.1% to 10% oxygen for a period of 30 minutes to 3 days. In some embodiments, regenerative fibroblast cells are exposed to 3% oxygen for 24 hours. In some embodiments, regenerative fibroblast cells are exposed to cobalt (II) chloride to chemically induce hypoxia. In some embodiments, regenerative fibroblast cells are exposed to cobalt (II) chloride for 1 to 48 hours. In some embodiments, regenerative fibroblast cells are exposed to cobalt (II) chloride for 24 hours. In some embodiments, regenerative fibroblast cells are exposed to 1 μM-300 μM. In some embodiments, regenerative fibroblast cells are exposed to 250 μM cobalt (II) chloride. In some embodiments, hypoxia induces an upregulation in HIF-1α, which is detected by expression of VEGF secretion. In some embodiments, hypoxia induces an upregulation of CXCR4 on fibroblast cells, which promotes homing of the cells to an SDF-1 gradient in inflamed areas.

In some embodiments, the method optionally includes enriching populations of fibroblast cells. In one embodiment, cells are transfected with a polynucleotide vector containing a stem cell-specific promoter operably linked to a reporter or selection gene. In some embodiments, the cell-specific promoter is an Oct-4, Nanog, Sox-9, GDF3, Rex-1, or Sox-2 promoter. In some embodiments, the method further includes the step of enriching the population for the regenerative fibroblast cells using expression of a reporter or selection gene. In some embodiments, the method further includes the step of enriching the population of the regenerative fibroblast cells by flow cytometry. In a further embodiment, the method further comprises the steps of selecting fibroblast cells expressing CD105 and/or CD 117 and transfecting the fibroblast cells expressing CD105 and/or CD 117 with cell-permanent NANOG gene.

In another embodiment, the method further includes the steps of contacting the fibroblast cells with a detectable compound that enters the cells, the compound being selectively detectable in proliferating and non-proliferating cells and enriching the population of cells for the proliferating cells. In some embodiments, the detectable compound is carboxyfluorescein diacetate, succinimidyl ester, or Aldefluor.

In some embodiments, fibroblast regenerative cells comprise fibroblast side population cells isolated based on expression of the multidrug resistance transport protein (ABCG2) or the ability to efflux intracellular dyes such as rhodamine-123 and or Hoechst 33342. Without being bound to theory, cells possessing this property express stem-like genes and are known for enhanced regenerative ability compared to cells which do not possess this efflux mechanism. Fibroblast side population cells are derived from tissues including pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, mesentery tissue, or a combination thereof.

### III. Generation of Regenerative Fibroblast-Conditioned Media

Certain aspects of the present disclosure relate to methods of reversing or substantially ameliorating the processes associated with ovarian failure through the therapeutic administration of concentrated media conditioned by regenerative fibroblast cells. In some embodiments, regenerative fibroblast cells are cultured in a growth medium to obtain conditioned media. In some embodiments, fibroblasts are cultured directly in tissue culture media including DMEM, EMEM, IMEM, or RPMI to produce fibroblast-conditoned media. In some embodiments, fibroblast-conditioned media is generated by culturing fibroblasts in hypoxic and/or hyperthermic conditions and/or with histone deacetylase inhibitors. In some embodiments, regenerative fibroblasts are also cultured alone or cultured in the presence of other cells to further upregulate production of growth factors in the conditioned media. Methods for generating conditioned media from fibroblasts are described herein.

Conditioned medium may be obtained from culture with fibroblasts. The cells may be cultured for about 1, 2, 3, 4, 5, 6, 7, 8, 9, 10 days or more. In some embodiments, the fibroblasts are cultured for about 3 days prior to collecting conditioned media. Conditioned media may be obtained by separating the cells from the media. Conditioned media may be centrifuged (e.g., at 500×g). Conditioned media may be filtered through a membrane. The membrane may be a >1000 kDa membrane. Conditioned media may be subject to liquid chromatography such as HPLC. Conditioned media may be separated by size exclusion.

Fibroblasts may be expanded and utilized by administration themselves, or may be cultured in a growth media in order to obtain conditioned media. The term Growth Medium generally refers to a medium sufficient for the culturing of fibroblasts. In particular, one presently preferred medium for the culturing of the cells herein comprises Dulbecco's Modified Essential Media (DMEM). Particularly preferred is DMEM-low glucose (also DMEM-LG herein) (Invitrogen®, Carlsbad, Calif.). The DMEM-low glucose is preferably supplemented with 15% (v/v) fetal bovine serum (e.g. defined fetal bovine serum, Hyclone™, Logan Utah), antibiotics/antimycotics (preferably penicillin (100 Units/milliliter), streptomycin (100 milligrams/milliliter), and amphotericin B (0.25 micrograms/milliliter), (Invitrogen®, Carlsbad, Calif.)), and 0.001% (v/v) 2-mercaptoethanol (Sigma®, St. Louis Mo.). In some cases different growth media are used, or different supplementations are provided, and these are normally indicated as supplementations to Growth Medium. Also relating to the present disclosure, the term standard growth conditions, as used herein refers to culturing of cells at 37° C., in a standard atmosphere comprising 5% CO₂, where relative humidity is maintained at about 100%. While the foregoing conditions are useful for culturing, it is to be understood that such conditions are capable of being varied by the skilled artisan who will appreciate the options available in the art for culturing cells, for example, varying the temperature, CO₂, relative humidity, oxygen, growth medium, and the like.

In one embodiment, the conditioned media comprises a liquid which has been in contact with fibroblast cells. In some embodiments, conditioned media is generated by culturing fibroblasts. In some embodiments, conditioned media is generated by combining fibroblasts with immune cells in a liquid media. Fibroblast cells may be fibroblasts that grow in an undifferentiated state and/or may be fibroblasts which have been stimulated with an agent that mimics inflammation and/or danger signals. Such agents are administered to fibroblasts in order to upregulate production of cytokines that are useful for the treatment of ovarian failure. In some embodiments cytokines secreted by fibroblasts whose upregulation is stimulated by exposure to inflammatory-related signals includes one or more of MCP-1, IL-6, IL-8, GCP-2, HGF, KGF, FGF, HB-EGF, BDNF, TPO, MIP1a, RANTES, TIMP1, HMGB1, IL-18, IL-33, or a combination thereof. In some embodiments fibroblasts are stimulated with agonists of toll-like receptors, including one or more of Triacyl lipopeptides, lipopeptides, lipoteichoic acid, HSP70, zymosan (Beta-glucan), double-stranded RNA, poly I:C, lipopolysaccharide, fibrinogen, heparan sulfate fragments, hyaluronic acid fragments, Bacterial flagellin, multiple diacyl lipopeptides, imidazoquinoline, loxoribine (a guanosine analogue), bropirimine, resiquimod, unmethylated CpG Oligodeoxynucleotide DNA, triacylated lipopeptides, Profilin, bacterial ribosomal RNA sequence “CGGAAAGACC,” or a combination thereof.

In one embodiment, provided is a means of creating a medicament useful for the treatment of ovarian failure by culturing cells in a serum free media. Many types of media may be chosen and used by one of skill in the art. In one embodiment, a media is selected from the group consisting of alpha MEM, DMEM, RPMI, Opti-MEM, IMEM, and AIM-V. Cells may be cultured in a variety of expansion media that contain fetal calf serum or other growth factors. However, for collection of therapeutic supernatant, in a specific embodiment, the cells are transferred to a media substantially lacking serum. In some embodiments, the supernatant is administered directly into the patient in need of treatment. It is well known in the art that preparation of the supernatant before administration may be performed by various means; for example, the supernatant may be filter sterilized or in some conditions concentrated. In a specific embodiment, the supernatant is administrated intramuscularly, intravenously, orally, sublingually, intranasally, intraventricularly, intrarectally, or intrathecally. Various volumes of injection may be used.

In some embodiments, culture conditioned media is concentrated by filtering/desalting means known in the art. In one embodiment, filters with specific molecular weight cut-offs are utilized. In one embodiments, the filters select for molecular weights between 1 kDa and 50 kDa. In one embodiment, the cell culture supernatant is concentrated using means known in the art such as solid phase extraction using C18 cartridges (Mini-Speed C18-14%, S.P.E. Limited, Concord ON). C18 cartridges are used to adsorb small hydrophobic molecules from the stem or progenitor cell culture supernatant, and allows for the elimination of salts and other polar contaminants. The cartridges are prepared by washing with methanol, followed by washing with deionized-distilled water. In some embodiments, up to 100 ml of stem cell or progenitor cell supernatant may be passed through each of these specific cartridges before elution, though one of skill in the art would understand that larger cartridges may be used. After washing the cartridges, adsorbed material is eluted with methanol, evaporated under a stream of nitrogen, redissolved in a small volume of methanol, and stored at 4° C. Before testing the eluate for activity in vitro, the methanol is evaporated under nitrogen and replaced by culture medium. In other embodiments, different adsorption means known in the art are used to purify certain compounds from fibroblast cell supernatants.

In some embodiments, further purification and concentration is performed using gel filtration with a Bio-Gel P-2 column having a nominal exclusion limit of 1800 Da (Bio-Rad, Richmond Calif.). The column is washed and pre-swelled in 20 mM Tris-HCl buffer, pH 7.2, (Sigma) and degassed by gentle swirling under vacuum. Bio-Gel P-2 material is packed into a 1.5×0.54 cm glass column and equilibrated with 3 column volumes of the same buffer. Cell supernatant concentrates extracted by filtration are dissolved in 0.5 ml of 20 mM Tris buffer, pH 7.2, and run through the column. Fractions are collected from the column and analyzed for biological activity. In alternative embodiments, other purification, fractionation, and identification means known to one skilled in the art including anionic exchange chromatography, gas chromatography, high performance liquid chromatography, nuclear magnetic resonance, and mass spectrometry are used to prepare concentrated supernatants.

In some embodiments, active supernatant fractions are administered locally or systemically. The supernatant concentrated from fibroblast-conditioned media is assessed directly for biological activities or further purified. In vitro bioassays allow for identification of the molecular weight fraction of the supernatant possessing biological activity and quantification of biological activity within the identified fractions. Production of various proteins and biomarkers associated is assessed by analysis of protein content using techniques including mass spectrometry, column chromatography, immune based assays such as enzyme linked immunosorbent assay (ELISA), immunohistochemistry, and flow cytometry.

### IV. Exosomes Purified from Fibroblasts

The present disclosure provides means of inhibiting and/or treating aneurysms and other degenerated blood vessels through administration of a fibroblast cell population and/or fibroblast-produced therapeutic factors. Fibroblast-produced therapeutic factors can include exosomes, apoptotic bodies, and/or microvesicles produced by fibroblasts, for example.

In some embodiments, exosomes purified from transfected fibroblasts are used therapeutically. In some embodiments, exosomes purified from fibroblast-conditioned media are used therapeutically. In some embodiments, purified fibroblast exosomes are used to regenerate functional tissue, induce proliferation, stimulate maturation of oocytes from oocyte progenitor cells, and/or induce folliculogenesis. In some embodiments, fibroblast-derived exosomes are used to suppress inflammation, including but not limited to suppressing production of IL-1, IL-6, and TNF-alpha by macrophages. Methods for purifying exosomes are known in the art and described herein.

In one embodiment, fibroblasts are cultured using means known in the art for preserving the viability and proliferative ability of fibroblasts. Both individualized autologous exosome preparations and exosome preparations obtained from established cell lines for experimental or biological use may be used. In one embodiment, chromatography separation methods are used to prepare membrane vesicles, particularly to separate the membrane vesicles from potential biological contaminants, wherein the membrane vesicles are exosomes and cells used to generate the exosomes are fibroblast cells.

In one embodiment, strong or weak anion exchange is performed. In addition, in a specific embodiment, the chromatography is performed under pressure. Thus, in some embodiments, the chromatography consists of high performance liquid chromatography (HPLC). Different types of column supports may be used to perform the anion exchange chromatography. In some embodiments, the column supports include cellulose, poly(styrenedivinylbenzene), agarose, dextran, acrylamide, silica, ethylene glycol-methacrylate co-polymer, or mixtures thereof, e.g., agarose-dextran mixtures. Column supports include but are not limited to gels including: SOURCE™, POROS™, SEPHAROSE™, SEPHADEX™ TRISACRYL™, TSK-GEL SW™ or PW™, SUPERDEX™, TOYOPEARL HW™, and SEPHACRYL™. Therefore, in a specific embodiment, membrane vesicles, particularly exosomes, are prepared from a biological sample such as a tissue culture containing fibroblasts, comprising at least one step during which the biological sample is purified by anion exchange chromatography on an optionally-functionalized column support selected from one or more of cellulose, poly(styrene-divinylbenzene), silica, acrylamide, agarose, dextran, ethylene glycol-methacrylate co-polymer, alone or in combinations thereof.

In some embodiments, the column supports are in bead form to improve chromatographic resolution. The beads can be homogeneous and calibrated in diameter with a sufficiently high porosity to enable the penetration of objects like exosomes undergoing chromatography. The diameter of exosomes is generally between 50 and 100 nm. Thus, in some embodiments, high porosity gels with diameters between about 10 nm and about 5 μm, about 20 nm and about 2 μm, or about 100 nm and about 1 μm are used. For anion exchange chromatography, the column support used can be functionalized with a group capable of interacting with an anionic molecule. Generally, this group is composed of a ternary or quaternary amine, which defines a weak or strong anion exchanger, respectively. In some embodiments, a strong anion exchanger corresponding to a chromatography column support functionalized with quaternary amines is used. Therefore, according to a more specific embodiment, anion exchange chromatography is performed on a column support functionalized with a quaternary amine and selected from one or more of poly(styrenedivinylbenzene), acrylamide, agarose, dextran, and silica, alone or in combinations thereof, and functionalised with a quaternary amine. Column supports functionalized with a quaternary amine include but are not limited to gels including SOURCE™ Q, MONO Q™ Q SEPHAROSE™, POROS™ HQ and POROS™ QE, FRACTOGEL™ TMAE type gels and TOYOPEARL SUPER™ Q gels.

In one embodiment, the column support used to perform the anion exchange chromatography comprises poly(styrene-divinylbenzene), for example, SOURCE Q gels like SOURCE™ 15Q (Pharmacia). This column support comprises large internal pores, low resistance to liquid circulation through the gel, and rapid diffusion of exosomes to the functional groups. Biological materials including exosomes retained on the column support may be eluted using methods known in the art, for example, by passing a saline solution gradient of increasing concentration over the column support. In some embodiments, a sodium chloride solution is used in concentrations varying from 0 to 2 M, for example. Purified fractions are detected based on optical densities (OD) of the fractions measured at the column support outlet using a continuous spectrophotometric reading. In some embodiments, fractions comprising membrane vesicles are eluted at an ionic strength of approximately 350 to 700 mM, depending on vesicle type.

Different types of chromatographic columns may be used depending on experimental requirements and volumes to be purified. For example, depending on the preparations, column volumes can vary from 100 μl up to >10 ml, and column supports can bind and retain up to 25 mg of proteins/ml. As an example, a 100 μl column has a capacity of approximately 2.5 mg of protein, which allows for purification of approximately 2 liters of culture supernatants concentrated by a factor of 10 to 20 to yield volumes of 100 to 200 ml per preparation. Higher volumes may also be purified by increasing the column volume.

Membrane vesicles can also be purified using gel permeation liquid chromatography. In some embodiments, the anion exchange chromatography step is combined with a gel permeation chromatography step either before or after the anion exchange chromatography step. In some embodiments, the permeation chromatography step takes place after the anion exchange step. In some embodiments, the anion exchange chromatography step is replaced with the gel permeation chromatography step. To perform gel permeation chromatography, a support selected from one or more of silica, acrylamide, agarose, dextran, ethylene glycol-methacrylate co-polymer, alone or in combinations thereof, e.g., agarose-dextran mixtures. Column supports include but are not limited to gels including SUPERDEX™ 200HR (Pharmacia), TSK G6000 (TosoHaas), and SEPHACRYL™ S (Pharmacia).

Gel permeation chromatography may be applied to different biological samples. In some embodiments, biological samples include but are not limited to biological fluid from a subject (bone marrow, peripheral blood, etc.), cell culture supernatant, cell lysate, pre-purified solution, or any other composition comprising membrane vesicles. In one embodiment, the biological sample is a culture supernatant of membrane vesicle-producing fibroblast cells treated, prior to chromatography, so as to enrich the supernatant for membrane vesicles. Thus, one embodiment relates to a method of preparing membrane vesicles from a biological sample, the method characterized by at least a) an enrichment step to prepare a sample enriched with membrane vesicles and b) a purification step during which the sample is purified by anion exchange chromatography and/or gel permeation chromatography.

In some embodiments, the biological sample is composed of an enriched, pre-purified solution obtained by centrifugation, clarification, ultrafiltration, nanofiltration and/or affinity chromatography of a cell culture supernatant of a membrane vesicle-producing fibroblast cell population or biological fluid. Thus, one embodiment relates to a method of preparing membrane vesicles comprising at least the steps of a) culturing a population of membrane vesicle-producing cells under conditions enabling the release of vesicles; b) enriching membrane vesicles in the sample; and c) performing anion exchange chromatography and/or gel permeation chromatography to purify the sample.

In some embodiments, the sample (e.g. supernatant) enrichment step comprises one or more centrifugation, clarification, ultrafiltration, nanofiltration, affinity chromatography, or a combination thereof. In one embodiment, the enrichment step comprises the steps of (i) elimination of cells and/or cell debris (clarification) and (ii) concentration and/or affinity chromatography. In one embodiment, affinity chromatography following clarification is optional. In one embodiment, the enrichment step comprises the steps of (i) elimination of cells and/or cell debris (clarification); (ii) concentration; and (iii) an affinity chromatography.

In some embodiments, the elimination step of enrichment is achieved by centrifugation of the sample, for example, at a speeds below 1000 g, such as between 100 and 700 g. In one embodiment, centrifugation conditions are approximately 300 g or 600 g for a period between 1 and 15 minutes.

In some embodiments, the elimination step of enrichment is achieved by filtration of the sample. In some embodiments, sample filtration is combined with centrifugation as described. The filtration may be performed with successive filtrations using filters with a decreasing porosity. In one embodiment, filters with a porosity between 0.2 and 10 μm are used. In one embodiment, a succession of filters with a porosities of 10 μm, 1 μm, 0.5 μm, and 0.22 μm are used.

In some embodiments, the concentration step of enrichment is performed to reduce the volume of sample to be purified during chromatography. In some embodiments, the concentration step of enrichment is achieved by centrifugation of the sample at speeds between 10,000 and 100,000 g to cause the sedimentation of the membrane vesicles. In some embodiments, the concentration step of enrichment is performed as a series of differential centrifugations, with the last centrifugation performed at approximately 70,000 g. After centrifugation, the pelleted membrane may be resuspended in a smaller volume of suitable buffer.

In some embodiments, the concentration step of enrichment is achieved by ultrafiltration which allows both to concentration of the supernatant and initial purification of the vesicles. In one embodiment, the biological sample (e.g., the supernatant) is subjected to tangential ultrafiltration consisting of concentration and fractionation of the sample between two compartments (filtrate and retentate) separated by membranes of determined cut-off thresholds. Separation of the sample is carried out by applying a flow in the retentate compartment and a transmembrane pressure between the retentate compartment and the filtrate compartment. Different systems may be used to perform the ultrafiltration, such as spiral membranes (Millipore, Amicon), flat membranes, or hollow fibers (Amicon, Millipore, Sartorius, Pall, GF, Sepracor). In some embodiments, membranes with cut-off thresholds below 1000 kDa, 300 kDa to 1000 kDa, or 300 kDa to 500 kDa are used.

The affinity chromatography step can be performed in various ways, using different chromatographic support and material known in the art. In some embodiments, non-specific affinity chromatography aimed at retaining (i.e., binding) certain contaminants present within the solution without retaining the objects of interest (i.e., the exosomes) is used as a form of negative selection. In some embodiments, affinity chromatography on a dye is used, allowing for the elimination (i.e., the retention) of contaminants such as proteins and enzymes like albumin, kinases, deshydrogenases, clotting factors, interferons, lipoproteins, or also co-factors, etc. The supports used for affinity chromatography on a dye are the supports used for ion exchange chromatography functionalized with a dye. In some embodiments, the dye is selected from the group consisting of Blue SEPHAROSE™ (Pharmacia), YELLOW 86, GREEN 5, and BROWN 10 (Sigma). In some embodiments, the support is agarose. However, those of skill in the art will understand that any other support and/or dye or reactive group allowing the retention (binding) of contaminants from the biological sample to be purified can be used.

Thus, one embodiment relates to a method of preparing membrane vesicles comprising the steps of a) culturing a population of membrane vesicle-producing cells under conditions enabling release of the vesicles; b) treating the culture supernatant with at least one ultrafiltration or affinity chromatography step to produce a biological sample enriched with membrane vesicles; and c) using anion exchange chromatography and/or gel permeation chromatography to purify the biological sample. In some embodiments, step b) comprises filtration of the culture supernatant, followed by an ultrafiltration, including tangential ultrafiltration. In further embodiments, step b) comprises clarification of the culture supernatant, followed by an affinity chromatography on dye, including Blue SEPHAROSE™.

In some embodiments, after step c), the harvested material is subjected to d) one or more additional treatment and/or filtration steps for sterilization purposes. For step d), filters with a diameter ≤0.3 μm or ≤0.25 μm are used. In some embodiments, after step d), the sterilized, purified material obtained is distributed into suitable containers such as bottles, tubes, bags, syringes, etc., in a suitable storage medium and stored cold, frozen, or used extemporaneously. Thus, in some embodiments, the method of preparing membrane vesicles further comprises c) anion exchange chromatography purification of the biological sample and d) a sterilizing filtration step of the material harvested in step c). In further embodiments, the method of preparing membrane vesicles further comprises c) gel permeation chromatography purification of the biological sample and d) a sterilizing filtration step of the material harvested in step c). In additional embodiments, the method of preparing membrane vesicles further comprises c) anionic exchange purification of the biological sample followed or preceded by gel permeation chromatography and d) a sterilizing filtration step of the material harvested in step c).

### V. Methods and Compositions for Treatment or Prevention of Ovarian Failure

Certain aspects of the present disclosure relate to the use of fibroblast cells and regenerative fibroblasts for treatment or prevention of ovarian failure in an individual. Methods for generation of regenerative fibroblasts are described elsewhere herein. In some embodiments, the disclosed methods comprise providing an effective amount of regenerative fibroblasts to an individual sufficient to treat ovarian failure. In some embodiments, regenerative fibroblasts may act to suppress fibrosis of the ovaries, inhibit inflammation, stimulate maturation of immature ovarian progenitor cells, or directly differentiate into oocytes, thereby treating or preventing ovarian failure in the individual. In some embodiments, regenerative fibroblasts produce factors that inhibit apoptosis of oocytes and/or oocyte progenitors to treat or prevent ovarian failure in the individual.

Disclosed in one embodiment is the generation of oocytes in an ovary which is not capable of producing oocytes by administration of fibroblasts expressing the marker CD39 and/or CD73. The ability of fibroblasts to stimulate production of oocytes can be enhanced by selecting fibroblasts expressing CD39 and/or CD73. In some embodiments, various factors may also be added to the culture to increase the number of fibroblasts expressing CD39 and/or CD73 and to augment the potency of these fibroblasts in stimulating oocyte production. For example, factors that could be utilized for augmentation of oogenic activity of regenerative fibroblasts include TPO, SCF, IL-1, IL-3, IL-6, IL-7, IL-11, flt-3L, G-CSF, GM-CSF, Epo, FGF-1, FGF-2, FGF-4, FGF-20, IGF, EGF, NGF, LIF, PDGF, BMPs, activin-A, VEGF, forskolin, and glucocorticoids.

As disclosed herein, a subpopulation of fibroblasts cells expresses CD39, CD73, and/or Oct3/4, a marker associated with an undifferentiated/pluripotent state. Oct3/4 is a POU-domain transcription factor associated with pluripotent stem cell capacity and is strongly expressed in female germ cells. Thus, in one embodiment of the method, a germ cell or a gamete-like cell, for example, an oocyte-like cell, is generated in vitro using the disclosed fibroblast-derived Oct3/4⁺ precursor cells. In another embodiment, an oocyte-like cell is generated that is Oct3/4⁺/Sca1⁻/CD34⁻/cKit⁺/Flk1⁺/FGFR⁺. The in vitro generated oocyte can then develop into a structure resembling a blastocyst in, for example, a petri dish without being fertilized (parthenogenesis).

Also disclosed, in one embodiment, are fibroblasts which are mitotically competent and express Oct 4, Vasa, Dazl, Stella, Fragilis, and optionally Nobox, c-Kit and Sca-1. Fibroblasts possessing these characteristics may be used for ovarian regeneration. Consistent with their mitotically competent phenotype, these fibroblasts do not express growth/differentiation factor-9 (“GDF-9”), zona pellucida proteins (e.g., zona pellucida protein-3, “ZP3”), histone deacetylase-6 (“HDAC6”) and synaptonemal complex protein-3 (“SCP3”). Upon transplantation into a host, these fibroblasts can produce oocytes after a duration of at least 1 week, 1 to about 2 weeks, about 2 to about 3 weeks, about 3 to about 4 weeks, or more than about 5 weeks post-transplantation.

In another embodiment, compositions comprising progenitor cells derived from fibroblasts treated with a histone deacetylase inhibitor are disclosed. The progenitor cells express Oct 4, Vasa, Dazl, Stella, Fragilis, and optionally Nobox, c-Kit and Sca-1 and do not express GDF-9, zona pellucida proteins, HDAC6 and SCP3. Upon transplantation into a host, the progenitor cells and/or fibroblasts of the disclosure can produce oocytes after a duration of less than 1 week, preferably about 24 to about 48 hours post transplantation.

In yet another embodiment, oocytes are produced by providing a fibroblast and/or a fibroblast progenitor cell to a tissue, for example, the ovary, wherein the fibroblast engrafts into the tissue and differentiates into an oocyte, thereby producing an oocyte.

In another embodiment, isolated fibroblasts are provided wherein the fibroblasts are mitotically competent and express Oct 4, Vasa, Dazl, Stella, Fragilis, and optionally Nobox, c-Kit and Sca-1. Preferably, the fibroblast has an XX karyotype.

Also disclosed in one embodiment are purified populations of fibroblasts that are derived so as to be capable of differentiating into female germline stem cells and/or germline progenitor cells. In specific embodiments, the purified population of cells is about 50 to about 55%, about 55 to about 60%, about 65 to about 70%, about 70 to about 75%, about 75 to about 80%, about 80 to about 85%, about 85 to about 90%, about 90 to about 95% or about 95 to about 100% of the cells in the composition.

In one embodiment, oocytes are produced from fibroblasts differentiated into female germline stem cells and/or germline progenitor cells cultured in the presence of an agent that differentiates the fibroblast-derived female germline stem cells and/or germline progenitor cells into an oocyte, thereby producing an oocyte. In a specific embodiment, the agent includes but is not limited to a hormone or growth factor (e.g., a TGF, BMP (bone morphogenic protein), or Wnt family protein, kit-ligand (“SCF”) or leukemia inhibitory factor (“LIF”)), a signaling molecule (e.g., meiosis-activating sterol, “FF-MAS”), or a pharmacologic or pharmaceutical agent (e.g., a modulator of Id protein function or Snail/Slug transcription factor function).

In one embodiment, provided are pharmaceutical compositions comprising fibroblast-derived female germline stem cells and/or germline progenitor cells and a pharmaceutically acceptable carrier. The pharmaceutical compositions can comprise purified populations of fibroblast-derived female germline stem cells and/or germline progenitor cells. Compositions comprising fibroblast-derived female germline stem cells can be provided by direct, local administration to ovarian tissue or indirect, systemic administration, for example, to the circulatory system of a subject (e.g., to the extra-ovarian circulation). In some embodiments, the fibroblasts may be generated from a variety of tissues including skin, placenta, omentum, adipose tissue, bone marrow, foreskin, umbilical cord, cord blood, amnion, amniotic fluid, embryos, hair follicle, nails, endometrium, keloids, ear lobe, testicles, Wharton's Jelly, and/or plastic surgery-related by-products.

In one embodiment, provided is a method for expanding fibroblast-derived female germline stem cells and/or germline progenitor cells in vivo, ex vivo, or in vitro. The fibroblasts are contacted with an agent that increases the amount of fibroblast-derived female germline stem cells and/or germline progenitor cells by promoting proliferation or survival thereof, thereby expanding fibroblast-derived female germline stem cells and/or germline progenitor cells. In a specific embodiment, the agent can include but is not limited to a hormone or growth factor (e.g., insulin-like growth factor (“IGF”), transforming growth factor (“TGF”), bone morphogenic protein (“BMP”), Wnt protein, or fibroblast growth factor (“FGF”)), a cell-signaling molecule (e.g., sphingosine-1-phosphate (“SIP”), or retinoic acid (“RA”)), or a pharmacological or pharmaceutical compound (e.g., an inhibitor of glycogen synthase kinase-3 (“GSK-3”), an inhibitor of apoptosis such as a Bax inhibitor or a caspase inhibitor, an inhibitor of nitric oxide production, or an inhibitor of HDAC activity). Other factors include TPO, SCF, IL-1, IL-3, IL-6, IL-7, IL-11, flt-3L, G-CSF, GM-CSF, Epo, FGF-1, FGF-2, FGF-4, FGF-20, IGF, EGF, NGF, LIF, PDGF, BMPs, activin-A, VEGF, forskolin, and glucocorticoids.

In another embodiment, provided is a method for identifying an agent that promotes proliferation or survival of a fibroblast-derived female germline stem cell and/or germline progenitor cell. The fibroblast-derived female germline stem cells or germline progenitor cells are contacted with a test agent. Subsequently, an increase in the number of fibroblast-derived female germline stem cells or germline progenitor cells is detected to identify an agent that promotes proliferation or survival of a fibroblast-derived female germline stem cell and/or germline progenitor cell.

In yet another embodiment, provided is a method for using the female germline stem cells and/or germline progenitor cells to characterize pharmacogenetic cellular responses to biologic or pharmacologic agents. Fibroblasts are isolated and expanded in culture to establish a plurality of cell cultures which can optionally be differentiated into a desired lineage. The cultured fibroblasts are contacted with one or more biologic or pharmacologic agents, and one or more cellular responses to the one or more biologic or pharmacologic agents are identified and compared with the cellular responses of fibroblasts cultured from different subjects.

In yet another embodiment, provided is a method for identifying an agent that induces differentiation of fibroblasts and/or fibroblast progenitor cells, into an oocyte comprising contacting bone marrow derived female germline stem cells, or their progenitor cells, with a test agent; and detecting an increase in the number of oocytes, thereby identifying an agent that induces differentiation of a bone marrow derived female germline stem cell, or its progenitor.

Also disclosed in one embodiment is a method for inducing folliculogenesis. A fibroblast-derived female germline stem cell and/or germline progenitor cell is provided to a tissue, for example, the ovary, wherein the cell engrafts into the tissue and differentiates into an oocyte within a follicle, thereby inducing folliculogenesis.

One embodiment provides a method of preserving fertility or treating infertility, for example, in subjects who require chemotherapy. A therapeutically effective amount of a composition comprising fibroblast-derived female germline stem cells and/or germline progenitor cells is administered to a subject, wherein the cells engraft into a tissue, for example, ovarian tissue, and differentiate into oocytes, thereby treating infertility. Except where expressly stated herein, the female subject in need of fertility treatment is not a subject who has undergone prior chemotherapy or radiotherapy.

In one embodiment, fibroblasts are used to generate T regulatory cells (Treg), which induce repair and/or regeneration of aged or damaged oocytes, oocyte progenitors, and/or the oocyte microenvironment. Without wishing to be bound by theory, it is known that Tregs cells are an essential component of the immune system as they protect the body against autoimmune attack. There are several subsets of Treg cells. Studies have found the Treg phenotype to include expression of CD4 and the IL-2 receptor CD25, which is also found on activated T cells. Peripheral blood contains a small population of T cell lymphocytes that express the Treg phenotype.

One subset of regulatory cells develops in the thymus. Thymic-derived Treg cells function by a cytokine-independent mechanism, which involves cell-to-cell contact. They are essential for the induction and maintenance of self-tolerance and for the prevention of autoimmunity. These regulatory cells prevent the activation and proliferation of autoreactive T cells that have escaped thymic deletion or recognize extrathymic antigens, thus they are critical for homeostasis and immune regulation, as well as for protecting the host against the development of autoimmunity. This is illustrated by early studies in which neonatally thymectomized mice suffered from systemic autoimmunity but were rescued by transfer of CD4 cells. Thus, immune regulatory CD4⁺CD25⁺T cells are often referred to as “professional suppressor cells.” Suppression of immunity by Treg cells occurs through several mechanisms. One is direct lysis of activated T cells, another one is inhibition of dendritic cell maturation, thus inhibiting ability of the antigen presenting cell arm of the immune system to initiate or perpetuate T cell responses.

Naturally-arising CD4⁺CD25⁺ Treg cells are a distinct population of cells that are positively selected on high affinity ligands in the thymus and that have been shown to play an important role in the establishment and maintenance of immunological tolerance to self-antigens. Deficiencies in the development and/or function of these cells have been associated with severe autoimmunity in humans and various animal models of congenital or induced autoimmunity.

In one embodiment, low dose interleukin-2 is provided to enhance Treg cells in vivo. Methods of utilizing interleukin-2 for stimulation of Treg cells in vivo is described are known in the art. For example, administration of interleukin-2 at doses below 5000 IU per kilogram may be performed in order to increase number of T regulatory cells. Alternatively, doses of interleukin 2 are used that do not stimulate effector T cell number but rather stimulate T regulatory cell activity and affect the number of T regulatory cells. In some embodiments, rapamycin, anti-CD3, and/or anti-CD45 RB, are used to increase Treg numbers in vivo subsequent to fibroblast administration. In some embodiments, Treg numbers are expanded in vivo subsequent to fibroblast administration in a patient suffering from ovarian failure through manipulation of the patient microbiome. In one embodiment, the activity of Treg cells that are generated in vitro or in vivo is further augmented by modulation of the host microbiome. Modulation of the microbiome may be performed by administration of either probiotics and/or prebiotics. It is known in the art that intestinal microbiota drives host immune homeostasis by regulating the differentiation and expansion of Treg, Th1, and Th2 cells. It has been demonstrated that FoxP3+ Treg cell deficiency results in gut microbial dysbiosis and autoimmunity. Means of remodeling the microbiome are described in the art and include administration of Lactobacillus reuteri, which is associated in animal models of autoimmunity with prolonged survival and reduced multi-organ inflammation. In one embodiment, L. reuteri is administered to change the metabolomic profile disrupted by Treg cell deficiency and to restore levels of the purine metabolite inosine. In one embodiment, administration of inosine together with the generation of Treg cells is used to stimulate enhanced suppressive activity and treatment of autoimmunity. Means of administering inosine and modulation of the microbiome for augmentation of Treg cells are known in the art. It has been reported that administration of inosine or administration of probiotics results in enhancement of T regulatory cell number and activity (He, B., et al., J Exp. Med., 214(1): 107-123 (2017)).

In another embodiment, fibroblasts treated with different agents such as TGF-β and/or HLA-G are used for in vitro or in vivo expansion of Treg cells. These agents can increase the expression of PD-1 in fibroblasts, which can result in the conversion of naïve T cells to Tregs upon binding of naïve T cells to fibroblasts expressing PD-1. Other techniques can be used to induce Treg generation in vitro using mesenchymal stem cells are known in the art and can be adapted by substituting mesenchymal stem cells with fibroblasts. For example, administration of mesenchymal stem cells together with probiotics is a means of stimulating ovarian function. Previous studies have demonstrated that mesenchymal stem cells (MSC) are capable of producing growth factors associated with cellular proliferation such as FGF, VEGF, IGF-1, and HGF. In fact, MSC feeder layers have previously been used to expand hematopoietic and pluripotent stem cells while maintaining these cells in an undifferentiated state. Furthermore, MSC have been demonstrated to promote generation of Treg cells in vitro and in vivo by modulating histone deacetylation (Azevedo, R., et al., Stem Cells, 2020:1-13). In one embodiment, fibroblasts are utilized as a substitute for MSC to induce Treg generation.

### VI. Administration of Therapeutic Compositions_{[NRF1]}

The therapy provided herein may comprise administration of a therapeutic agents (e.g., fibroblasts, exosomes from fibroblasts, other fibroblast-derived products or fibroblast-produced therapeutic factors, etc.) alone or in combination. Therapies may be administered in any suitable manner known in the art. For example, a first and second treatment may be administered sequentially (at different times) or concurrently (at the same time). In some embodiments, the first and second treatments are administered in a separate composition. In some embodiments, the first and second treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents (e.g., fibroblasts) of the disclosure may be administered by the same route of administration or by different routes of administration. In some embodiments, the cancer therapy is administered intravenously, intramuscularly, subcutaneously, topically, orally, sublingually, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

In some embodiments, subjects are treated with fibroblasts within a composition. Fibroblasts can be activated prior to therapeutic use and/or fibroblasts can be administered with agents which act as “regenerative adjuvants” for the fibroblasts. In some embodiments, compositions containing fibroblasts comprise between about 10,000-10 million cells per kilogram of body weight. In some embodiments, approximately 1 million cells per kilogram of body weight are administered. In some embodiments, between about 10⁵ and about 10¹³ cells per 100 kg are administered to a human per infusion. In some embodiments, between about 1.5×10⁶ and about 1.5×10¹² cells are infused per 100 kg. In some embodiments, between about 1×10⁹ and about 5×10¹¹ cells are infused per 100 kg. In some embodiments, between about 4×10⁹ and about 2×10¹¹ cells are infused per 100 kg. In some embodiments, between about 5×10⁸ cells and about 1×10¹¹ cells are infused per 100 kg. In some embodiments, between about 50 million and about 500 million fibroblast cells are administered to the subject. For example, between about 50 million and about 100 million fibroblast cells, between about 50 million and about 200 million fibroblast cells, between about 50 million and about 300 million fibroblast cells, between about 50 million and about 400 million fibroblast cells, between about 100 million and about 200 million fibroblast cells, between about 100 million and about 300 million fibroblast cells, between about 100 million and about 400 million fibroblast cells, between about 100 million and about 500 million fibroblast cells, between about 200 million and about 300 million fibroblast cells, between about 200 million and about 400 million fibroblast cells, between about 200 million and about 500 million fibroblast cells, between about 300 million and about 400 million fibroblast cells, between about 300 million and about 500 million fibroblast cells, between about 400 million and about 500 million fibroblast cells, about 50 million fibroblast cells, about 100 million fibroblast cells, about 150 million fibroblast cells, about 200 million fibroblast cells, about 250 million fibroblast cells, about 300 million fibroblast cells, about 350 million fibroblast cells, about 400 million fibroblast cells, about 450 million fibroblast cells, or about 500 million fibroblast cells may be administered to the subject.

In some embodiments, a single administration of cells is provided. The cells can be fibroblasts and/or other cells of the disclosure. In some embodiments, cells are administered once monthly, weekly, and/or daily. In some embodiments, multiple administrations are provided. In some embodiments, multiple administrations are provided over the course of 3-7 consecutive days. In some embodiments, 3-7 administrations are provided over the course of 3-7 consecutive days. In some embodiments, 5 administrations are provided over the course of 5 consecutive days. In some embodiments, a single administration of between about 10⁵ and about 10¹³ cells per 100 kg is provided. In some embodiments, a single administration of between about 1.5×10⁸ and about 1.5×10¹² cells per 100 kg is provided. In some embodiments, a single administration of between about 1×10⁹ and about 5×10¹¹ cells per 100 kg is provided. In some embodiments, a single administration of about 5×10¹⁰ cells per 100 kg is provided. In some embodiments, a single administration of 1×10¹⁰ cells per 100 kg is provided. In some embodiments, multiple administrations of between about 10⁵ and about 10¹³ cells per 100 kg are provided. In some embodiments, multiple administrations of between about 1.5×10⁸ and about 1.5×10¹² cells per 100 kg are provided. In some embodiments, multiple administrations of between about 1×10⁹ and about 5×10¹¹ cells per 100 kg are provided over the course of 3-7 consecutive days. In some embodiments, multiple administrations of about 4×10⁹ cells per 100 kg are provided over the course of 3-7 consecutive days. In some embodiments, multiple administrations of about 2×10¹¹ cells per 100 kg are provided over the course of 3-7 consecutive days. In some embodiments, 5 administrations of about 3.5×10⁹ cells are provided over the course of 5 consecutive days. In some embodiments, 5 administrations of about 4×10⁹ cells are provided over the course of 5 consecutive days. In some embodiments, 5 administrations of about 1.3×10¹¹ cells are provided over the course of 5 consecutive days. In some embodiments, 5 administrations of about 2×10¹¹ cells are provided over the course of 5 consecutive days.

The quantity to be administered, both according to number of treatments and unit dose, depends on the treatment effect desired. An effective dose is understood to refer to an amount necessary to achieve a particular effect. In some embodiments, it is contemplated that doses between about 10⁵ and about 10¹³ cells per 100 kg are administered to affect the protective capability of the fibroblasts. In certain embodiments, it is contemplated that a cell number in the range from 10 mg/kg to 200 mg/kg can affect the protective capability of these agents. Thus, it is contemplated that doses include doses of cell numbers of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In some embodiments, it is contemplated that a dose of a drug or other therapeutic agent in the range from 10 mg/kg to 200 mg/kg can affect the protective capability of these agents. Thus, it is contemplated that doses include doses of drugs or other therapeutic agents of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain embodiments, the effective dose of a pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

### VII. Kits of the Disclosure

Any of the cellular and/or non-cellular compositions described herein or similar thereto may be comprised in a kit. In a non-limiting example, one or more reagents for use in methods for preparing fibroblasts or derivatives thereof (e.g., exosomes derived from fibroblasts) may be comprised in a kit. Such reagents may include cells, vectors, one or more growth factors, vector(s) one or more costimulatory factors, media, enzymes, buffers, nucleotides, salts, primers, compounds, and so forth. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

In specific embodiments, reagents and materials include primers for amplifying desired sequences, nucleotides, suitable buffers or buffer reagents, salt, and so forth, and in some cases the reagents include apparatus or reagents for isolation of a particular desired cell(s).

In particular embodiments, there are one or more apparatuses in the kit suitable for extracting one or more samples from an individual. The apparatus may be a syringe, fine needles, scalpel, and so forth.

### EXAMPLES

The following examples are included to demonstrate particular embodiments of the invention. It should be appreciated by those of skill in the art that the techniques disclosed in the examples that follow represent techniques discovered by the inventors to function well in the practice of the methods of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Fibroblast Administration into Chemotherapy Treated Mice Normalizes Hormonal Production in a Manner Superior to Bone Marrow Mesenchymal Stem Cells

Adult female C57BL6 mice, 4 to 6 weeks of age, weighing between 20 and 25 g, were purchased from Charles River Co (Wilmington, Mass.). Animals were housed in groups of 6 within polyethylene cages and allowed to acclimatize to the animal facility environment for at least 1 week prior to study initiation. Animals were maintained in an environmentally controlled room with 22° C. with a humidity range of 50% to 60%, 12-hour dark-12-hour light cycles (lights on at 6:00 AM). Animals had free and continuous access to water and commercial pelleted mouse chow.

At random, acclimatized mice (N=6/group) were assigned to a control group (group 1); a chemotherapy group with sham stem cell treatment (group 2); a bone marrow mesenchymal stem cell-based treatment after chemotherapy group (group 3); and a fibroblast cell treated group (Group 4). Group 1 was subjected to bilateral intraovarian administration of 10 μL phosphate-buffered saline/ovary via laparotomy. Chemotherapy (CTX) combination treatment consisted of a single intraperitoneal injection of busulfan (12 mg/kg) and cyclophosphamide (70 mg/kg), both dissolved in saline as described previously and confirmed by the manufacturer (Sigma Aldrich, USA). This combination is hereafter referred to as CTX. Seven days post-CTX injection, mice in groups 2, 3, and 4 were subjected to bilateral intraovarian administration of 10 μL, phosphate-buffered saline via laparotomy, 10 μL of bone marrow mesenchymal stem cell (BMSC) suspension (5×10⁵ cells), or 10 μL of fibroblast suspension (5×10⁵ cells) in each ovary, respectively. In order to administer the cells, a 10-μL, G30 Hamilton syringe (Harvard apparatus, MA, USA) was used; the fascia and skin were closed using vicryl 3 (ETHICON USA) zero sutures. Aseptic procedures were used at all times.

The BMSCs were purchased from Allcells at passage 2 and plated in vitro per the manufacturer's instructions for 1 passage to confirm viability. Fibroblasts were obtained from dermal biopsy of human skin, expanded in OPTI-MEM media, and selected for CD73 expression using magnetic activated cell sorting (MACS). Cells were collected at room temperature and suspended in 10 μL of phosphate-buffered saline. BMSCs and fibroblast cells were immediately injected into both ovaries (right and left) of each animal in groups 3 and 4, respectively, at a concentration of 5×10⁵ cells per ovary.

In a first set of experiments, time points were set at 2, 4, and 6 weeks from the day of surgery. At each time point, animals (N=6/group) hormonal assays were conducted. Moreover, 1 animal from each group was killed, and ovarian tissues were excised from each animal (N=6/group) and fixed in 4% buffered formalin for 24 hours prior to storage (in 70% ethanol) until paraffin blocked and sectioned. Ovarian tissue sections (5-μm thick) were subsequently subjected to hematoxylin and eosin (H&E) staining to assess the distribution of ovarian follicular developmental stage and antral follicle size. Suppression of chemotherapy-induced FSH production was more potent by administration of fibroblasts compared to administration of BMSCs.

In a second set of experiments, at random, acclimatized mice (N=6/group) were assigned to a control group (group 1″), sham chemotherapy group (group 2″), bone marrow stem cell-based treatment after chemotherapy (group 3″), or fibroblast cell-based treatment after chemotherapy (group 4″) as aforementioned; animal cohabitation was initiated 1 week after surgical recovery with age- and strain-matched breeder males at a ratio of 2 females:1 male. Fruitful mating was determined by the presence of plugs in the vaginal os of females. All pups were gathered, counted, and examined closely for weight as well as any evident congenital anomalies or abnormal physical findings.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A method of treating or preventing ovarian failure in an individual comprising administering a therapeutically effective amount of a composition comprising fibroblasts or conditioned media therefrom to an individual in need thereof.

2. The method of claim 1, wherein the ovarian failure is age-related.

3. The method of claim 1, wherein the ovarian failure is idiopathic premature ovarian failure.

4. The method of claim 1, wherein the ovarian failure is associated with treatment.

5. The method of claim 4, wherein the treatment is chemotherapy, radiation therapy, or a combination thereof.

6. The method of claim 1, wherein the fibroblasts comprise regenerative fibroblasts.

7. The method of claim 1, wherein the fibroblast cells are cultured under conditions sufficient to differentiate the fibroblasts into regenerative fibroblast cells.

8. The method of claim 6 or 7, wherein the regenerative fibroblast cells comprise one or more of the following biological activities:
(a) inducing of angiogenesis;
(b) producing trophic factors;
(c) suppressing inflammation;
(d) stimulating maturation of immature oocytes; and
(e) inducing folliculogenesis.

9. The method of any one of claims 6-8, wherein the regenerative fibroblast cells are cultured under conditions sufficient to enhance the ability of the regenerative fibroblast cells to induce angiogenesis, produce trophic factors, suppress inflammation, stimulate maturation of immature oocytes, induce folliculogenesis, or a combination thereof.

10. The method of any one of claims 7-9, wherein the conditions comprise hypoxia.

11. The method of claim 10, wherein the hypoxia is sufficient to induce nuclear translocation of HIF-1 alpha.

12. The method of any one of claims 7-11, wherein the conditions further comprise treatment of the regenerative fibroblast cells with one or more growth factors, one or more differentiation factors, one or more dedifferentiation factors, or a combination thereof.

13. The method of any one of claims 6-12, wherein the regenerative fibroblast cells express one or more markers selected from the group consisting of Oct-4, Nanog, Sox-2, KLF4, c-Myc, Rex-1, GDF-3, LIF receptor, CD105, CD117, CD344, Stella, and a combination thereof.

14. The method of any one of claims 6-13, wherein the regenerative fibroblast cells do not express one or more cell surface proteins selected from the group consisting of MHC class I, MHC class II, CD45, CD13, CD49c, CD66b, CD73, CD105, CD90, and a combination thereof.

15. The method of any one of claims 6-14, wherein the regenerative fibroblast cells have enhanced GDF-11 expression compared to a control or standard.

16. The method of any one of claim 1 or 6-15, wherein the fibroblast cells are, or are derived from, fibroblasts isolated from umbilical cord, skin, cord blood, adipose tissue, hair follicle, omentum, bone marrow, peripheral blood, Wharton's Jelly, or a combination thereof.

17. The method of any one of claim 1 or 6-16, wherein the fibroblast cells are obtained from dermal fibroblasts, placental fibroblasts, adipose fibroblasts, bone marrow fibroblasts, foreskin fibroblasts, umbilical cord fibroblasts, hair follicle derived fibroblasts, nail derived fibroblasts, endometrial derived fibroblasts, keloid derived fibroblasts, or a combination thereof.

18. The method of any one of claim 1 or 6-17, wherein the fibroblast cells are autologous, allogeneic, or xenogeneic to the recipient.

19. The method of any one of claim 1 or 6-18, wherein the fibroblast cells are purified from bone marrow.

20. The method of any one of claim 1 or 6-18, wherein the fibroblast cells are purified from peripheral blood.

21. The method of any one of claims 6-20, wherein the regenerative fibroblast cells are isolated from peripheral blood of an individual who has been exposed to one or more conditions and/or one or more therapies sufficient to stimulate regenerative fibroblast cells from the individual to enter the peripheral blood of the individual.

22. The method of claim 21, wherein the conditions sufficient to stimulate regenerative fibroblast cells from the individual to enter the peripheral blood comprise administration of G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA reductase inhibitors, small molecule antagonists of SDF-1, or a combination thereof.

23. The method of claim 21 or 22, wherein the therapies sufficient to stimulate regenerative fibroblast cells from the individual to enter the peripheral blood comprise therapies including exercise, hyperbaric oxygen, autohemotherapy by ex vivo ozonation of peripheral blood, induction of SDF-1 secretion in an anatomical area outside of the bone marrow, or a combination thereof.

24. The method of any one of claims 6-23, wherein the regenerative fibroblast cells are comprised of an enriched population of regenerative fibroblast cells.

25. The method of claim 24, wherein enrichment is achieved by:
(a) transfecting the cells with a vector comprising a fibroblast-specific promoter operably linked to a reporter or selection gene, wherein the reporter or selection gene is expressed, and
(b) enriching the population of cells for cells expressing the reporter or selection gene.

26. The method of claim 24 or 25, wherein enrichment is achieved by:
(a) treating the cells with a detectable compound, wherein the detectable compound is selectively detectable in proliferating and non-proliferating cells, and
(b) enriching the population of cells for proliferating cells.

27. The method of claim 26, wherein the detectable compound is selected from a group comprising carboxyfluorescein diacetate, succinimidyl ester, and Aldefluor.

28. The method of any one of claims 6-27, wherein the regenerative fibroblast cells are fibroblasts isolated as side population cells.

29. The method of claim 28, wherein the fibroblasts isolated as side population cells are identified based on expression of the multidrug resistance transport protein (ABCG2).

30. The method of claim 28 or 29, wherein the fibroblasts isolated as side population cells are identified based on the ability to efflux intracellular dyes.

31. The method of claims 28-30, wherein the side population cells are derived from tissues selected from the group consisting of pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, mesentery tissue, and a combination thereof.

32. The method of any of claims 6-31, wherein the fibroblast cells express CD39.

33. The method of any of claims 6-32, wherein the fibroblast cells express CD73.

34. The method of claims 33, wherein the CD73-positive fibroblast cells are cultured under hypoxic conditions.

35. The method of claim 34, wherein the hypoxic conditions comprise from 0.1% oxygen to 10% oxygen for a period of 30 minutes to 3 days.

36. The method of claim 34, wherein the hypoxic conditions comprise 3% oxygen for 24 hours.

37. The method of claim 34, wherein hypoxic conditions are chemically induced.

38. The method of claim 37, wherein chemical induction of hypoxia comprises culture in cobalt (II) chloride.

39. The method of claim 38, wherein fibroblast cells are cultured with 1 μM-300 μM cobalt (II) chloride.

40. The method of claim 39, wherein the fibroblast cells are incubated with 250 μM of cobalt (II) chloride.

41. The method of claims 39 and 40, wherein the fibroblast cells are further cultured for 1-48 hours.

42. The method of claim 41, wherein the fibroblast cells are cultured for a time period of 24 hours.

43. The method of claim 34-42, wherein the hypoxic conditions induce upregulation of HIF-1α.

44. The method of claim 43, wherein expression of HIF-1α is detected by expression of VEGF secretion.

45. The method of claim 34-44, wherein the hypoxic conditions induce upregulation of CXCR4 on the fibroblast cells.

46. The method of claim 45, wherein upregulation of CXCR4 promotes homing of the fibroblast cells to an SDF-1 gradient.

47. The method of any of claims 6-46, wherein the fibroblast cells express Oct3/4.

48. The method of claims 6-47, wherein the fibroblast cells are treated with a histone deactylase inhibitor.

49. The method of claim 48, wherein the histone deacetylase inhibitor is selected from a group consisting of sodium butyrate, valproic acid, and trichostatin A.

50. The method of claims 32-49, wherein the fibroblast cells are induced in culture to express Vasa, Dazl, Stella, Fragilis, or a combination thereof.

51. The method of any of claims 32-50, wherein the fibroblast cells do not express GDF-9, zona pellucida proteins, HDAC6, SCP3, or a combination thereof.

52. The method of claims 32-51, wherein the fibroblast cells are mitotically competent.

53. The method of claims 32-52, wherein the fibroblast cells possess an XX karyotype.

54. The method of claims 1-53, wherein an individual with ovarian failure is treated with fibroblasts to increase the number and activity of T regulatory cells, and wherein the fibroblast cells are capable of inducing generation of T regulatory cells.

55. The method of claim 54, wherein the fibroblast cells capable of inducing generation of T regulatory cells produce growth factors.

56. The method of claim 55, wherein the growth factors comprise FGF, VEGF, IGF-1, HGF, or a combination thereof.

57. The method of claims 54-56, wherein the T regulatory cells express the transcription factor FoxP3.

58. The method of claims 54-57, wherein the T regulatory cells express the transcription factor Helios.

59. The method of claims 54-58, wherein the T regulatory cells suppress fibrosis.

60. The method of claims 54-59, wherein the T regulatory cells produce interleukin-10.

61. The method of claims 54-60, wherein the T regulatory cells produce interleukin-35.

62. The method of claims 54-61, wherein activity of the T regulatory cells is augmented by manipulation of the individual's microbiome.

63. The method of claim 62, wherein manipulation of the individual's microbiome is by administration of Lactobacillus reuteri.

64. The method of claim 54-63, further comprising administration of inosine.

65. The method of claims 1-64, wherein the fibroblast cells are cultured with one or more agents capable of increasing expression of fibroblast PD-1 ligand.

66. The method of claim 65, wherein the agents comprise TGF-β and/or soluble HLA-G.

67. The method of claim 65 or 66, wherein the increased expression of fibroblast PD-1 ligand is associated with induction of T regulatory cells upon binding of PD-1-expressing fibroblasts to naïve T cells.

68. The method of claims 1-67, wherein the fibroblast cells induce generation of adaptive immune cells, and wherein the adaptive immune cells are capable of reprogramming ovarian cells to suppress inflammation.

69. The method of claim 68, wherein the adaptive immune cell is a B regulatory cell.

70. The method of claim 68, wherein the adaptive immune cell is a T regulatory cell.

71. The method of claim 70, wherein the T regulatory cells express CD4 and CD25.

72. The method of any of claims 1-71, wherein the fibroblast cells are administered locally or systemically.

73. The method of claim 72, wherein local administration is inside the ovary, in the periovary area, or a combination thereof.

74. The method of any of claims 1-73, wherein administration of fibroblast cells stimulates production of oocytes.

75. The method of claim 74, wherein the stimulation of oocyte production by fibroblast cells is augmented by culture with factors including TPO, SCF, IL-1, IL-3, IL-6, IL-7, IL-11, flt-3L, G-CSF, GM-CSF, Epo, FGF-1, FGF-2, FGF-4, FGF-20, IGF, EGF, NGF, LIF, PDGF, BMPs, activin-A, VEGF, forskolin, glucocorticoids, or a combination thereof.

76. The method of any one of claims 1-75, further defined as administering to the individual a therapeutically effective amount of a composition comprising regenerative fibroblast-conditioned media.

77. The method of claim 76, wherein regenerative fibroblast cells are cultured under conditions sufficient to upregulate production of one or more growth factors in the regenerative fibroblast-conditioned media.

78. The method of claims 76 and 77, wherein the regenerative fibroblast-conditioned media is concentrated.

79. The method of claims 76-78, wherein the regenerative fibroblast-conditioned media is administered locally or systemically to the individual.

80. A method of oocyte production comprising culturing isolated fibroblast cells in the presence of an agent that differentiates the fibroblast cells into an oocyte, thereby producing an oocyte.

81. The method of claim 80, wherein the agent is selected from the group consisting of transforming growth factor, bone morphogenic protein, Wnt family protein, kit-ligand, leukemia inhibitory factor, meiosis-activating sterol, modulator of Id protein function, and modulator of Snail/Slug transcription factor function.

82. A method of oocyte production comprising administering fibroblast-derived germline cells to an individual, wherein the cells engraft into a tissue and differentiate into an oocyte, thereby producing an oocyte.

83. The method of claim 82, wherein fibroblast-derived germline cells are obtained by culturing fibroblasts with transforming growth factor, bone morphogenic protein, Wnt family protein, kit-ligand, leukemia inhibitory factor, meiosis-activating sterol, modulator of Id protein function, modulator of Snail/Slug transcription factor function, or a combination thereof.

84. The method of claim 82, wherein the fibroblast-derived germline cells are germline stem cells.

85. The method of claim 84, wherein the fibroblast-derived germline cells are germline progenitor cells.

86. A method of inducing folliculogenesis comprising administering fibroblast-derived germline cells to an ovary, wherein the cells engraft into the ovary and differentiate into an oocyte within a follicle.

87. The method of claim 86, wherein the fibroblast-derived germline cells are germline stem cells.

88. The method of claim 86, wherein the fibroblast-derived germline cells are germline progenitor cells.

89. A pharmaceutical composition comprising a purified population of cells that are mitotically competent, have an XX karyotype and express Vasa, Dazl and Stella.

90. The pharmaceutical composition of claim 89, wherein the purified population of cells are capable of differentiating into female germline cells.

91. The pharmaceutical composition of claim 90, wherein the female germline cells are female germline progenitor cells.

92. The pharmaceutical composition of claim 90, wherein the female germline cells are female germline stem cells.

93. The pharmaceutical composition of claims 89-92, wherein the cells are purified from fibroblasts.

94. The pharmaceutical composition of claim 93, wherein the cells are mammalian cells.

95. The pharmaceutical composition of claim 94, wherein the cells are human cells.

96. The pharmaceutical composition of claims 89-95, wherein the purified population of cells is about 50 to about 55%, about 55 to about 60%, about 65 to about 70%, about 70 to about 75%, about 75 to about 80%, about 80 to about 85%, about 85 to about 90%, about 90 to about 95% or about 95 to about 100% of the cells in the composition.

97. The pharmaceutical composition of claims 89-96, further comprising a pharmaceutically acceptable carrier.

98. The pharmaceutical composition of claims 89-97, wherein the composition is administered locally or systemically.

99. The pharmaceutical composition of claim 98, wherein local administration is inside the ovary, in the peri-ovary area, or a combination thereof.
